(19) United States
(12) Patent Application Publication
(10) Pub. No.: US 2024/0198142 A1
(43) Pub. Date: Jun. 20, 2024

(54) ELECTRONIC MASK AND CONTROLLING METHOD THEREOF

(71) Applicant: Samsung Electronics Co., Ltd., Suwon-si (KR)

(72) Inventors: Hyojin KIM, Suwon-si (KR); Yeonseung OH, Suwon-si (KR); Sungjune CHO, Suwon-si (KR); Younglae JO, Suwon-si (KR)

(21) Appl. No.: 18/516,870

(22) Filed: Nov. 21, 2023

Related U.S. Application Data
(63) Continuation of application No. PCT/KR2023/014233, filed on Sep. 20, 2023.

(30) Foreign Application Priority Data
Dec. 20, 2022 (KR) 10-2022-0179870

(51) Int. Cl. A62B 18/00; A61B 5/08; A61M 16/06; A62B 7/10; A62B 18/02

(52) U.S. Cl. CPC A62B 18/006; A61B 5/08; A61M 16/06; A62B 7/10; A62B 18/025; A61B 2562/0247; A61M 2230/40

(57) ABSTRACT

An electronic mask includes a memory configured to store default atmospheric pressure information corresponding to a user and a plurality of preset events corresponding to the default atmospheric pressure information, a pressure sensor configured to measure atmospheric pressure inside the electronic mask, a fan module configured to introduce outside air to inside of the electronic mask or discharge inside air to outside of the electronic mask, and at least one processor configured to obtain the measured atmospheric pressure through the pressure sensor, identify an event among the plurality of preset events based on the measured atmospheric pressure, and control the fan module to perform an operation corresponding to the identified event.

### CROSS-REFERENCE TO RELATED APPLICATION

This application is a bypass continuation of International Application No. PCT/KR2023/014233, filed on Sep. 20, 2023, which is based on and claims priority to Korean Patent Application No. 10-2022-0179870, filed on Dec. 20, 2022, in the Korean Intellectual Property Office, the disclosures of which are incorporated by reference herein in their entireties.

### BACKGROUND

### 1. Field

The disclosure relates to an electronic mask and a controlling method thereof and, more particularly to, a powered mask to automatically control a fan module and a controlling method thereof.

### 2. Description of Related Art

A powered mask operated by fan power to filter inhalation and exhalation according to breathing may be used.

In case of a mask for health, which is used to protect respiratory organs from particulate harmful substances such as yellow dust, substances for preventive measures, fine dust, and the like, and an infectious agent, the higher the efficiency of the filter, the worse for a user to breath.

In order to overcome this disadvantage, an electronic mask may control flow of air by applying a filter made of fiber and a filtering device using fan power.

In order to turn on/off the electronic mask or adjust the air volume, a user may directly press a button with the hand. When the mask is pressed, pressure is applied to the face and the user may feel inconvenience.

In addition, when a user cannot use both hands, the user may have a difficulty in directly pressing an operation button.

In addition, since the operation button of the electronic mask is not within the field of view of the user, the user may feel inconvenience as the user had to take off the mask to press the operation button or press the operation button while seeing a mirror.

### SUMMARY

The disclosure is designed to improve the above-described problem, and the purpose of the disclosure is to provide an electronic mask to automatically perform an operation corresponding to a preset event through measured atmospheric pressure without directly touching a mask and a method for controlling thereof.

An electronic mask according to various embodiments includes a memory configured to store default atmospheric pressure information corresponding to a user and a plurality of preset events corresponding to the default atmospheric pressure information, a pressure sensor configured to measure atmospheric pressure inside the electronic mask, a fan module configured to introduce outside air to inside of the electronic mask or discharge inside air to outside of the electronic mask, and at least one processor configured to obtain the measured atmospheric pressure through the pressure sensor, identify an event among the plurality of preset events based on the measured atmospheric pressure, and control the fan module to perform an operation corresponding to the identified event.

In certain embodiments, the default atmospheric pressure information may include a default minimum atmospheric pressure and a default maximum atmospheric pressure corresponding to the user, the at least one processor may obtain a first event atmospheric pressure less than the default minimum atmospheric pressure, obtain a second event atmospheric pressure greater than the default maximum atmospheric pressure, and identify the event by comparing the measured atmospheric pressure with at least one of the first event atmospheric pressure and the second event atmospheric pressure.

In certain embodiments, the least one processor may, in response to an intensity of the fan module changing, change the default minimum atmospheric pressure, the default maximum atmospheric pressure, or at least one of the first event atmospheric pressure and the second event atmospheric pressure based on the changed intensity of the fan module.

In certain embodiments, the at least one processor may, based on the measured atmospheric pressure being less than or equal to the first event atmospheric pressure, identify an inhalation event among the plurality of preset events.

In certain embodiments, the at least one processor may obtain a first event time at which the measured atmospheric pressure is less than or equal to the first event atmospheric pressure, based on the first event time being less than a threshold time, identify the inhalation event as a first type, and based on the first event time being greater than or equal to the threshold time, identify the inhalation event as a second type.

In certain embodiments, the at least one processor may, based on the measured atmospheric pressure being greater than or equal to the second atmospheric pressure, identify an exhalation event among the plurality of preset events.

In certain embodiments, the at least one processor may obtain a second event time at which the measured atmospheric pressure is greater than or equal to the second event atmospheric pressure, based on the second event time being less than a threshold time, identify the exhalation event as a first type, and based on the second event time being greater than or equal to the threshold time, identify the exhalation event as a second type.

In certain embodiments, the at least one processor may count a number of events based on an amount that the event is identified during a preset time, and identify the event based on the number of events.

In certain embodiments, the default atmospheric pressure information may include a default breathing time, and the at least one processor may, based on an inhalation event being identified, obtain a breathing time of the user based on a first time interval corresponding to an extreme minimum point, based on an exhalation event being identified, obtain a breathing time of the user based on a second time interval corresponding to an extreme maximum point, and identify the event based on comparing the default breathing time and the breathing time.

In certain embodiments, the electronic mask may include a communication interface, the at least one processor may receive a user input to change setting information of the electronic mask from a terminal device through the communication interface, and change setting information of the electronic mask based on the received user input.

A method of controlling an electronic mask storing default atmospheric pressure information corresponding to a user and a plurality of preset events corresponding to the default atmospheric pressure information according to various embodiments includes obtaining a measured atmospheric pressure through a pressure sensor of the electronic mask, identifying an event among the plurality of preset events based on the measured atmospheric pressure, and performing an operation corresponding to the identified event.

In certain embodiments, the default atmospheric pressure information may include a default minimum atmospheric pressure and a default maximum atmospheric pressure corresponding to the user, identifying the event may include obtaining a first event atmospheric pressure less than the default minimum atmospheric pressure, obtaining a second event atmospheric pressure greater than the default maximum atmospheric pressure, and identifying the event by comparing the measured atmospheric pressure with at least one of the first event atmospheric pressure and the second event atmospheric pressure.

The control method may further include, in response to an intensity of the fan module changing, changing the default minimum atmospheric pressure, the default maximum atmospheric pressure, or at least one of the first event atmospheric pressure and the second event atmospheric pressure based on the changed intensity of the fan module.

The identifying the event may include, based on the measured atmospheric pressure being less than or equal to the first event atmospheric pressure, identifying an inhalation event among the plurality of preset events.

The identifying the event may include obtaining a first event time at which the measured atmospheric pressure is less than or equal to the first event atmospheric pressure, based on the first event time being less than a threshold time, identifying the inhalation event as a first type, and based on the first event time being greater than or equal to the threshold time, identifying the inhalation event as a second type.

The identifying the event may include, based on the measured atmospheric pressure being greater than or equal to the second atmospheric pressure, identifying an exhalation event among the plurality of preset events.

The identifying the event may include obtaining a second event time at which the measured atmospheric pressure is greater than or equal to the second event atmospheric pressure, based on the second event time being less than a threshold time, identifying the exhalation event as a first type, and based on the second event time being greater than or equal to the threshold time, identifying the exhalation event as a second type.

In certain embodiments, the identifying the event may include identifying a number of events based on an amount that the event is identified during a preset time, and identifying the event based on the number of events.

The default atmospheric pressure information may include a default breathing time, and identifying the event may include, based on an inhalation event being identified, obtaining a breathing time of the user based on a first time interval corresponding to an extreme minimum point, based on an exhalation event being identified, obtaining the breathing time of the user based on a second time interval corresponding to an extreme maximum point, and identifying the event based on comparing the default breathing time and the breathing time.

The control method may include receiving a user input to change setting information of the electronic mask from a terminal device, and changing setting information of the electronic mask based on the received user input.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 is a diagram illustrating an electronic mask and a terminal device.

FIG. 2 is a block diagram illustrating an electronic mask according to various embodiments of the disclosure.

FIG. 3 is a block diagram illustrating a specific configuration of the electronic mask of FIG. 3.

FIG. 4 is a diagram illustrating a structure of an electronic mask according to various embodiments.

FIG. 5 is a diagram illustrating a structure of an electronic mask according to various embodiments.

FIG. 6 is a flowchart illustrating an operation of storing default atmospheric pressure information.

FIG. 7 is a diagram illustrating an operation of storing default atmospheric pressure information.

FIG. 8 is a flowchart illustrating an operation of identifying a preset event by using measured atmospheric pressure information.

FIG. 9 is a flowchart illustrating an operation of identifying breathing pattern information based on measured atmospheric pressure information.

FIG. 10 is a flowchart illustrating a plurality of normal modes.

FIG. 11 is a diagram illustrating a state of an electronic mask.

FIG. 12 is a diagram illustrating an embodiment in which a short exhalation event is identified once.

FIG. 13 is a diagram illustrating an embodiment in which a short exhalation event is identified twice.

FIG. 14 is a diagram illustrating an embodiment in which a short exhalation event is identified thrice.

FIG. 15 is a diagram illustrating an embodiment in which a long exhalation event is identified.

FIG. 16 is a diagram illustrating an embodiment in which a short inhalation event is identified once.

FIG. 17 is a diagram illustrating an embodiment in which a short inhalation event is identified twice.

FIG. 18 is a diagram illustrating an embodiment in which a short inhalation event is identified thrice.

FIG. 19 is a diagram illustrating an embodiment in which a long inhalation event is identified.

FIG. 20 is a flowchart illustrating an embodiment of identifying a preset event through a terminal device.

FIG. 21 is a flowchart illustrating an embodiment of changing a mask setting through a terminal device.

FIG. 22 is a diagram illustrating an operation of controlling a mask setting through an application.

FIG. 23 is a flowchart illustrating an embodiment in which default atmospheric pressure information and event atmospheric pressure information are differently applied according to a performance mode.

FIG. 24 is a diagram illustrating an embodiment in which default atmospheric pressure information is differently applied according to a performance mode.

FIG. 25 is a diagram illustrating an embodiment of differently applying event atmospheric pressure information according to a performance mode.

FIG. 26 is a flowchart illustrating an operation of correcting measurement atmospheric pressure information according to a performance mode.

FIG. 27 is a diagram illustrating a preset event (breathing pattern) corresponding to a state or mode.

FIG. 28 is a diagram illustrating a preset event (voice) corresponding to a state or mode.

FIG. 29 is a flowchart illustrating an operation of controlling an electronic mask through a user voice.

FIG. 30 is a flowchart illustrating an embodiment of analyzing a user voice by using a terminal device.

FIG. 31 is a flowchart illustrating a method for controlling an electronic mask according to various embodiments.

### DETAILED DESCRIPTION

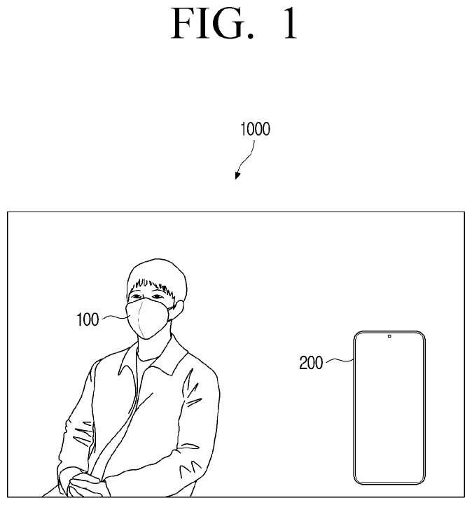

FIGS. 1 through 31, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged system or device.

The disclosure will be described in greater detail with reference to the attached drawings.

The terms used in the disclosure and the claims are general terms identified in consideration of the functions of embodiments of the disclosure. However, these terms may vary depending on intention, legal or technical interpretation, emergence of new technologies, and the like of those skilled in the related art. In addition, in some cases, a term may be selected by the applicant, in which case the term will be described in detail in the description of the corresponding disclosure. Thus, the term used in this disclosure should be defined based on the meaning of term, not a simple name of the term, and the contents throughout this disclosure.

Expressions such as “have,” “may have,” “include,” “may include” or the like represent presence of corresponding numbers, functions, operations, or parts, and do not exclude the presence of additional features.

Expressions such as “at least one of A or B” and “at least one of A and B” should be understood to represent “A,” “B” or “A and B.”

As used herein, terms such as “first,” and “second,” may identify corresponding components, regardless of order and/or importance, and are used to distinguish a component from another without limiting the components.

In addition, a description that one element (e.g., a first element) is operatively or communicatively coupled with/to” or “connected to” another element (e.g., a second element) should be interpreted to include both the first element being directly coupled to the second element, and the first element being indirectly coupled to the second element through a third element.

A singular expression includes a plural expression, unless otherwise specified. It is to be understood that terms such as “comprise” or “consist of” are used herein to designate a presence of a characteristic, number, step, operation, element, component, or a combination thereof, and not to preclude a presence or a possibility of adding one or more of other characteristics, numbers, steps, operations, elements, components or a combination thereof.

A term such as “module,” “unit,” and “part,” is used to refer to an element that performs at least one function or operation and that may be implemented as hardware or software, or a combination of hardware and software. Except when each of a plurality of “modules,” “units,” “parts,” and the like must be realized in an individual hardware, the components may be integrated in at least one module or chip and be realized in at least one processor.

In the following description, a “user” may refer to a person using an electronic mask or an electronic apparatus using an electronic mask (e.g., artificial intelligence electronic mask).

An embodiment of the disclosure will be described in more detail with reference to the accompanying drawings.

FIG. 1 is a diagram illustrating the electronic mask 100 and the terminal device 200.

A system 1000 of FIG. 1 may include at least one of an electronic mask 100 and a terminal device 200. The electronic mask 100 may mean a powered mask. The electronic mask 100 may refer to a mask that performs a function of physically blocking fine dust or viruses using power supplied to the electronic mask 100. The electronic mask 100 may include a fan for a blowing function and may drive a fan through the power supplied to the electronic mask 100. When the fan is driven, the electronic mask 100 may discharge air from inside the electronic mask 100 to the outside or introduce air from outside the electronic mask 100 into the electronic mask 100.

The electronic mask 100 may be described as a wearable mask, a powered mask, or the like.

The terminal device 200 may be a device connected to the electronic mask 100. For example, the terminal device 200 may be a smartphone, a tablet, and a wearable display device.

The electronic mask 100 and the terminal device 200 may communicatively connect. According to various embodiments, the electronic mask 100 and the terminal device 200 may be connected by using 1:1 or 1:n Bluetooth communication.

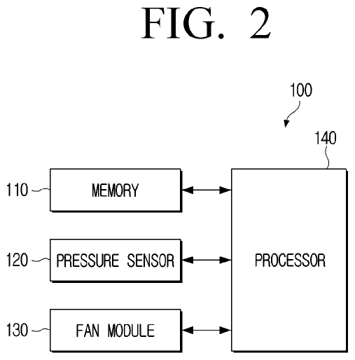

FIG. 2 is a block diagram illustrating the electronic mask 100 according to various embodiments of the disclosure.

Referring to FIG. 2, the electronic mask 100 may include at least one of a memory 110, a pressure sensor 120, a fan module 130, and at least one processor 140.

The electronic mask 100 may be used as a personal device rather than a public device due to a hygiene issue. Therefore, for the electronic mask 100, settings tailored to each individual user may be applied. The electronic mask 100 may store default (or standard or basis or normal) atmospheric pressure information of a user.

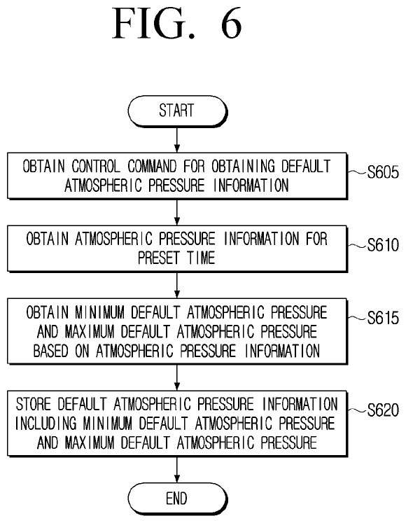

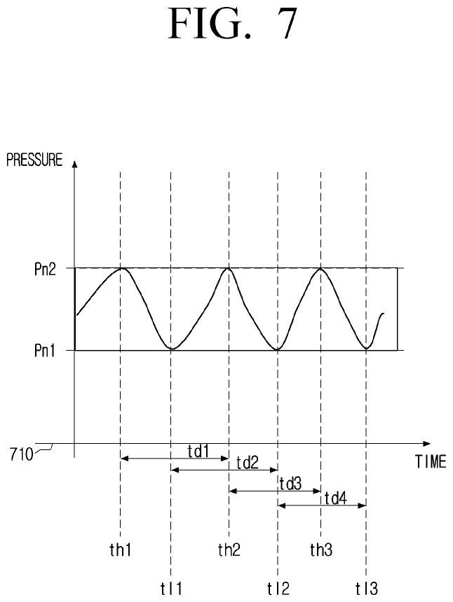

The electronic mask 100 may perform a process of registering (or generating) default atmospheric pressure information in a reset to factory settings. A specific description will be given in FIGS. 6 and 7.

In addition, the electronic mask 100 may store information about a preset event related to default atmospheric pressure information in the memory 110. The preset event may be changed in response to default atmospheric pressure information. For example, when a criterion for determining a preset event is an event atmospheric pressure the event atmospheric pressure may vary based on the default atmospheric pressure information.

The memory 110 may store default atmospheric pressure information corresponding to a user and a plurality of preset events corresponding to default atmospheric pressure information.

The pressure sensor 120 may sense and measure atmospheric pressure inside the electronic mask 100. The pressure sensor 120 may sense and measure a pressure of air inside the electronic mask 100. The pressure sensor 120 may be recited as an atmospheric pressure sensor, an atmospheric pressure sensor, an atmospheric pressure sensing module, or an atmospheric pressure measurement device. The pressure sensor 120 may described as the atmospheric pressure sensor.

The pressure sensor 120 may be a sensor to sense and measure air pressure inside the electronic mask 100. The pressure sensor 120 may be a sensor for generating atmospheric pressure data.

The fan module 130 may introduce external air into the electronic mask 100 or discharge air inside from an inside of the electronic mask 100 to the outside of the electronic mask 100. According to various embodiments, the electronic mask 100 may include a fan. The electronic mask 100 may perform an inhalation function and an exhalation function through the fan.

According to various embodiments, the electronic mask 100 may include a first fan and a second fan. The electronic mask 100 may perform an inhalation function through the first fan. The electronic mask 100 may perform an exhalation (or a discharge) function through a second fan.

At least one processor 140 may obtain a measured atmospheric pressure through the pressure sensor 120, and when one of a plurality of preset events is identified based on the measured atmospheric pressure, may control the fan module 130 to perform an operation corresponding to the identified event.

The at least one processor 140 may measure current atmospheric pressure inside the electronic mask 100 through the pressure sensor 120. The measured atmospheric pressure may be written as measured atmospheric pressure or measured atmospheric pressure information.

At least one processor 140 may identify a preset event based on the measured atmospheric pressure. The at least one processor 140 may identify a preset event among a plurality of preset events stored in the memory 110 based on the measured atmospheric pressure. One preset event may be described as an event.

The at least one processor 140 may perform a control operation of the electronic mask 100 based on an event identified according to measured atmospheric pressure. At least one processor 140 may perform an operation or function corresponding to the identified event.

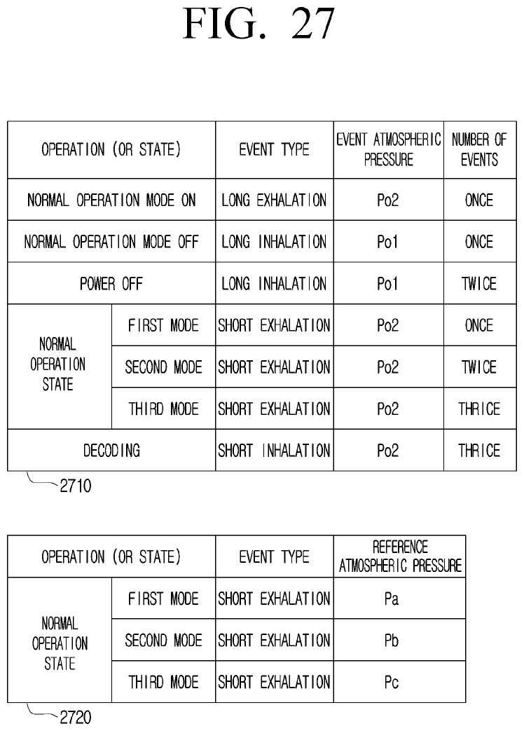

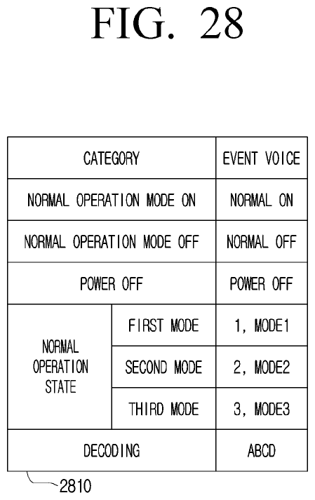

A specific detail related to an operation corresponding to an event will be described in FIGS. 27 and 28. The event identified as the measured atmospheric pressure will be described in FIG. 27. An event identified by a user voice will be described in FIG. 28.

At least one processor 140 may obtain or collect measured atmospheric pressure for a preset period. The information related to measured atmospheric pressure obtained during a preset period may be recited as measured atmospheric pressure information. The measured atmospheric pressure information may be information indicating measured atmospheric pressure according to a time.

In certain embodiments, default atmospheric pressure information may include a default minimum atmospheric pressure and a default maximum atmospheric pressure corresponding to a user, at least one processor 140 may obtain a first event atmospheric pressure less than the default minimum atmospheric pressure, obtain a second event atmospheric pressure greater than the default maximum atmospheric pressure, and may identify an event by comparing the measured atmospheric pressure with at least one of the first event atmospheric pressure and the second event atmospheric pressure.

The default atmospheric pressure information may include information about a default breathing pattern of a user. The default atmospheric pressure information may include at least one of a default minimum atmospheric pressure and a default maximum atmospheric pressure from normal breathing.

The at least one processor 140 may obtain a measured atmospheric pressure according to the breathing of the user for a preset time. It is assumed that the user performs normal breathing during a preset time period. The at least one processor 140 may determine the maximum atmospheric pressure among the measured atmospheric pressure measured for a preset time as a default maximum atmospheric pressure. Also, the at least one processor 140 may determine a minimum atmospheric pressure among measured atmospheric pressure measured for a preset time as a default minimum atmospheric pressure.

At least one processor 140 may obtain default atmospheric pressure information including default minimum atmospheric pressure and default maximum atmospheric pressure. At least one processor 140 may store the obtained default atmospheric pressure information in the memory 110.

A specific description related to the default atmospheric pressure information will be described in FIGS. 6 and 7.

At least one processor 140 may obtain event information based on default atmospheric pressure information. The event information may include a condition necessary to identify an event or an operation corresponding to an event. The event information may include at least one of an event type, an event atmospheric pressure, the number of events, and an operation (or state) corresponding to the event. A detailed description related thereto will be described in FIG. 27.

The event atmospheric pressure may mean a threshold atmospheric pressure (or atmospheric pressure value) to identify occurrence of a specific event.

At least one processor 140 may obtain a first event atmospheric pressure less than a default minimum atmospheric pressure. Specifically, at least one processor 140 may determine, as a first event atmospheric pressure, an atmospheric pressure multiplied by a threshold ratio (for example, 0.9) of a default minimum atmospheric pressure. Meanwhile, the threshold ratio may be changed according to a user setting.

The first event atmospheric pressure may be an inhalation event atmospheric pressure. The first event atmospheric pressure may be a reference atmospheric pressure used to identify an inhalation event. At least one processor 140 may compare a measured atmospheric pressure with a first event atmospheric pressure to identify whether a preset event has occurred. For example, when a measured atmospheric pressure equal to or less than the first event atmospheric pressure is identified, the at least one processor 140 may identify that an inhalation event has occurred.

The at least one processor 140 may obtain a second event atmospheric pressure greater than the default maximum atmospheric pressure. Specifically, at least one processor 140 may determine, as a second event atmospheric pressure, an atmospheric pressure multiplied by a threshold ratio (e.g., 0.9) of the default maximum atmospheric pressure. Meanwhile, the threshold ratio may be changed according to a user setting.

The second event atmospheric pressure may be an exhalation event atmospheric pressure. The second event atmospheric pressure may be a reference atmospheric pressure used to identify the exhalation event. At least one processor 140 may compare a measured atmospheric pressure with a second event atmospheric pressure to identify whether a preset event has occurred. For example, when the measured atmospheric pressure is identified as greater than or equal to the second event atmospheric event, the at least one processor 140 may identify that an exhalation event has occurred.

In certain embodiments, at least one processor 140 may, when the intensity of the fan module 130 changes, at least one of default minimum atmospheric pressure, default maximum atmospheric pressure, first event atmospheric pressure, and second event atmospheric pressure may be changed based on the changed intensity of the fan module 130.

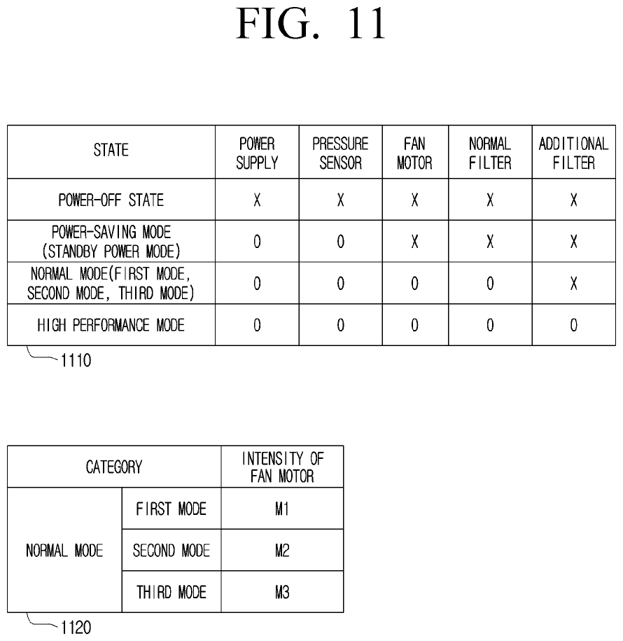

The fan module 130 may be controlled according to the mode or state of the electronic mask 100. The fan module 130 may differently drive the intensity of the motor based on the mode or state being performed. For example, as in the table 1120 of FIG. 11, the intensity of the fan motor may be different depending on the mode. In a first mode, the fan motor may be driven with a first intensity, in a second mode, the fan motor is driven with a second intensity, and, in a third mode, the fan motor may be driven with a third intensity.

It is assumed that the second intensity is greater than the first intensity, and the third intensity is greater than the second intensity. The intensity of the motor may mean the number of rotations of a motor per minute. As the number of rotations of the motor is larger (higher), the strength of the blowing may be increased. When the strength of the blowing increases, the pressure of air may be increased. Even in a breathing state of the same user, an atmospheric pressure obtained through the pressure sensor 120 may increase when the strength of the blowing is increased. Therefore, there may be a need to change a reference related to atmospheric pressure according to the intensity of the blowing (or the intensity of the fan module).

The at least one processor 140 may change the reference atmospheric pressure based on the intensity of the fan module 130.

According to various embodiments, when the intensity of the fan module 130 is changed, the at least one processor 140 may change default atmospheric pressure information based on the intensity of the fan module 130. When default atmospheric pressure information changes, at least one processor 140 may change a first event atmospheric pressure and a second event atmospheric pressure corresponding to default atmospheric pressure information.

According to various embodiments, when the intensity of the fan module 130 changes, at least one processor 140 may change the first event atmospheric pressure and the second event atmospheric pressure.

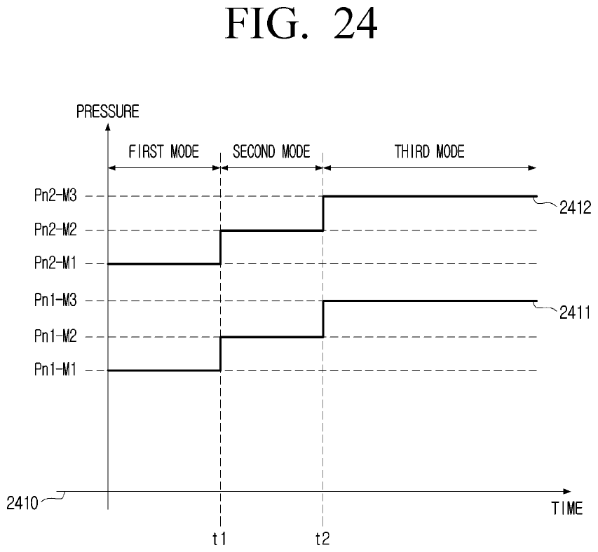

An embodiment of changing the default minimum atmospheric pressure and the default maximum atmospheric pressure will be described in FIG. 24.

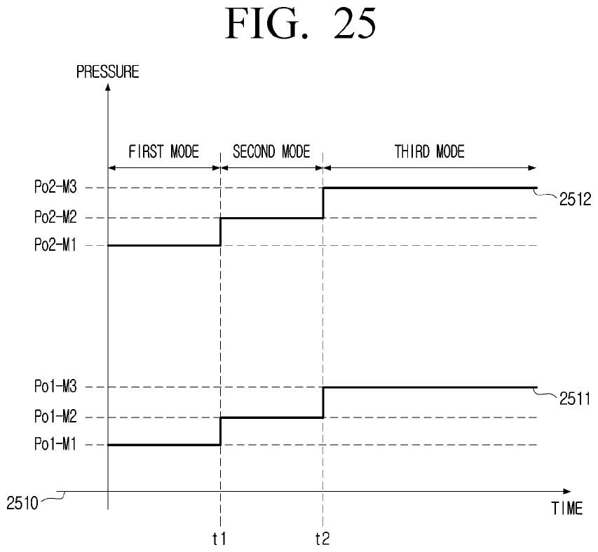

An embodiment of changing the first event atmospheric pressure and the second event atmospheric pressure will be described in FIG. 25.

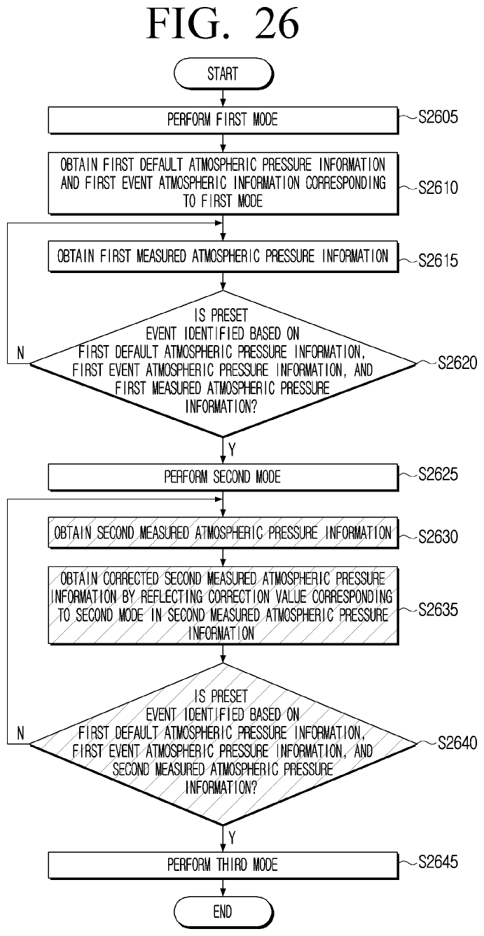

According to various embodiments, when the intensity of the fan module 130 changes, at least one processor 140 may change a measured atmospheric pressure. The at least one processor 140 may identify a correction value or a correction coefficient corresponding to the intensity of the fan module 130. The at least one processor 140 may obtain a corrected measured atmospheric pressure by applying a correction value at a measured atmospheric pressure. At least one processor 140 may compare the corrected measured atmospheric pressure and the event atmospheric pressure to identify a preset event. An embodiment related to the same will be described in FIG. 26.

In certain embodiments, at least one processor 140 may, if the measured atmospheric pressure is less than or equal to the first event atmospheric pressure, identify the inhalation event among a plurality of preset events.

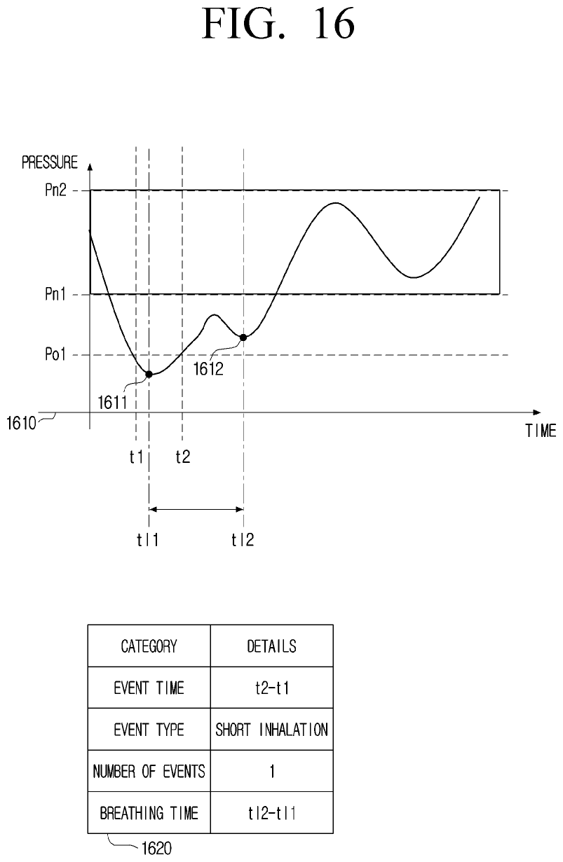

A specific description related to the inhalation event will be described in FIGS. 16 to 19. The first event atmospheric pressure may be Po1 described in FIGS. 16 to 19.

In certain embodiments, the at least one processor 140 may obtain a first event time at which a measured atmospheric pressure is sensed to be equal to or less than a first event atmospheric pressure, identify an inhalation event of a first type if the first event time is less than a threshold time, and identify an inhalation event of a second type if the first event time is greater than or equal to a threshold time.

In certain embodiments, when the inhalation event of the first type is identified, at least one processor 140 may perform an operation (or function) corresponding to the inhalation event of the first type.

In addition, when the inhalation event of the second type is identified, at least one processor 140 may perform an operation (or function) corresponding to the inhalation event of the second type.

At least one processor 140 may determine, as the first event time, a time at which the measured atmospheric pressure is obtained as being less than or equal to the first event atmospheric pressure during a preset period. The first event time may mean a time at which the atmospheric pressure consecutively measured is obtained as being less than or equal to the first event atmospheric pressure.

At least one processor 140 may identify the type of an inhalation event by comparing a first event time and a threshold time. The inhalation event may include an inhalation event of a first type and an inhalation event of a second type. An event time used to identify an inhalation event of a first type may be less than an event time used to identify an inhalation event of a second type.

The inhalation event of the first type may be described as a short inhalation. The inhalation event of a second type of may be described as a long inhalation. The expression “short” or “long” may be written as “first type” or “second type”. In addition, the expression “short” or “long” may be for relative comparison among an inhalation event of a first type and an inhalation event of a second type. Meanwhile, the threshold time may be changed according to a user setting.

In certain embodiments, at least one processor 140 may, when the measured atmospheric pressure is greater than or equal to the second event atmospheric pressure, identify an exhalation event among a plurality of preset events.

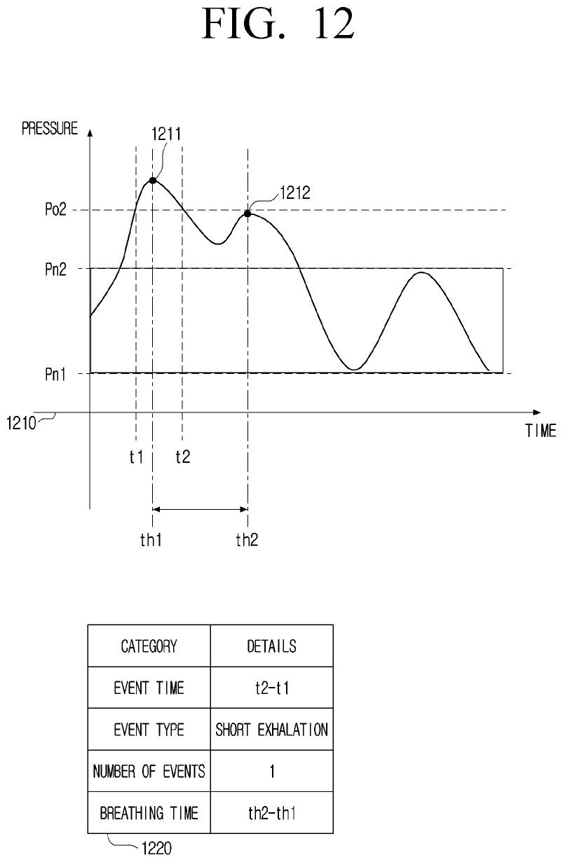

A specific description related to the exhalation event will be described in FIGS. 12 to 15. The second event atmospheric pressure may be Po2 value described in FIGS. 12 to 15.

In certain embodiments, at least one processor 140 may obtain a second event time at which a measured atmospheric pressure is sensed to be greater than or equal to a second event atmospheric pressure, identify an exhalation event of a first type if the second event time is less than a threshold time, and identify an exhalation event of a second type if the second event time is greater than or equal to a threshold time.

When the exhalation event of the first type is identified, at least one processor 140 may perform an operation or function corresponding to the exhalation event of the first type.

In addition, when the exhalation event of the second type is identified, at least one processor 140 may perform an operation or function corresponding to the exhalation event of the second type.

At least one processor 140 may determine, as a second event time, a time when a measured atmospheric pressure is obtained to be equal to or greater than a second event atmospheric pressure for a preset period. The second event time may mean a time when the consecutively measured atmospheric pressure is obtained to be equal to or greater than the second event atmospheric pressure.

At least one processor 140 may compare a second event time and a threshold time to identify a type of the exhalation event. The exhalation event may include a first type of exhalation event and a second type of exhalation event. The event time used to identify the exhalation event of the first type may be less than an event time used to identify an exhalation event of a second type.

The exhalation event of the first type may be described as a short exhalation. The exhalation event of the second type may be described as a long exhalation. The expression “short” or “long” may be written as “first type” or “second type”. In addition, the expression “short” or “long” may be for relative comparison among an exhalation event of a first type and an exhalation event of a second type. Meanwhile, the threshold time may be changed according to a user setting.

In certain embodiments, the at least one processor 140 may identify a number of events based on an amount that the event is identified for a preset time, and may identify an event based on the number of events.

At least one processor 140 may determine whether a specific event type is repeatedly identified for a preset time.

For example, an inhalation event of a first type may be repeatedly identified for a preset time. It may be one event that the inhalation event of the first type is identified for a plurality of times. The preset event may include an event in which an inhalation event of a first type is identified once, an event in which the inhalation event of the first type is identified twice, and an event in which the inhalation event of the first type is identified three thrice.

A specific description related to an event will be described in FIG. 27. An embodiment in which an inhalation event repeats once to three times will be described in FIGS. 16 to 18. An embodiment in which the exhalation event repeats once to thrice will be described in FIGS. 12 to 14.

In certain embodiments, default atmospheric pressure information includes a default breathing time, and at least one processor 140 may obtain a breathing time of a user based on a time interval corresponding to the extreme minimum point when an inhalation event is identified, obtain a breathing time of the user based on a time interval corresponding to the extreme maximum point when an exhalation event is identified, and may identify an event by comparing the default breathing time and the breathing time.

The default breathing time may mean breathing cycle of a user. A related description will be provided in FIG. 7.

The at least one processor 140 may compare a default breathing time pre-stored in the memory 110 with a measured breathing time sensed in real time. If the default breathing time and the measured breathing time are the same, the at least one processor 140 may determine that the event is not identified.

Even when there is no control intention according to the breathing pattern of the user, it may be determined that a preset event is identified according to the measured atmospheric pressure. If a specific operation is automatically performed differently from the user intention, the user may feel inconvenience.

Therefore, if default breathing time and the measured breathing time are different, at least one processor 140 may determine that the event, identified according to the measured atmospheric pressure, is definitely identified.

If the default breathing time and the measured breathing time are the same, at least one processor 140 may determine that an event, identified according to the measured atmospheric pressure, is not identified.

An embodiment in which user breathing time is obtained by using an extreme minimum point in an inhalation event will be described in FIGS. 16 to 18.

An embodiment in which user breathing time is obtained by using an extreme maximum point in an exhalation event will be described in FIGS. 12 to 14.

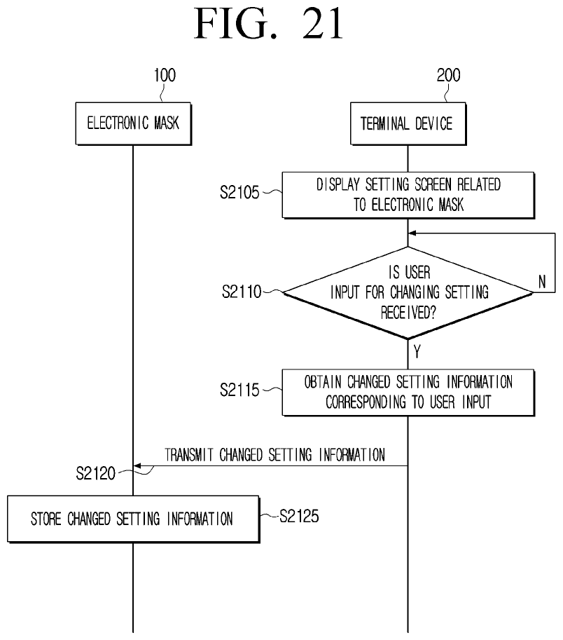

In certain embodiments, the electronic mask 100 may include a communication interface 160, and the at least one processor 140 may receive, from the terminal device 200, a user input for changing configuration information of the electronic mask 100 through the communication interface, and change configuration information of the electronic mask 100 based on the received user input. A detailed description related to the same is described in FIG. 21.

In certain embodiments, the electronic mask 100 according to various embodiments may automatically control a detailed function of the electronic mask 100 according to a breathing pattern of a user. Even when a user does not use a remote control device or a terminal device 200 in a state of wearing the electronic mask 100, the user may easily control the electronic mask 100 by only an input related to breathing of the user.

In addition, a user may easily change various setting (e.g., reference atmospheric pressure, etc.) applicable to the electronic mask 100 through an application, or the like, installed in the terminal device 200.

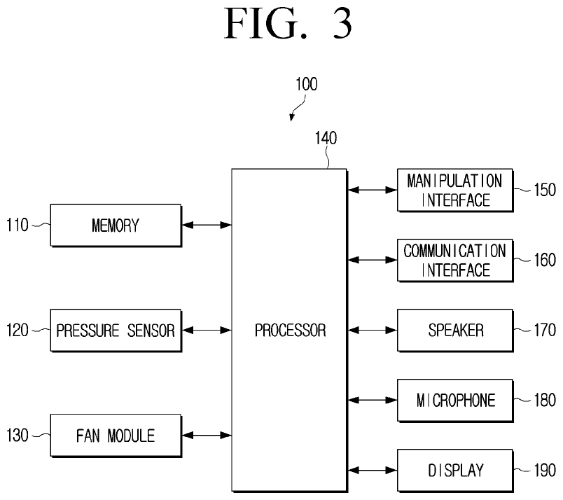

It has been described a simple configuration constituting the electronic mask 100 above, but in implementation, various configurations may be additionally provided. This will be described below with reference to FIG. 3.

FIG. 3 is a block diagram illustrating a specific configuration of the electronic mask of FIG. 3.

Referring to FIG. 3, the electronic mask 100 may include at least one of the memory 110, the pressure sensor 120, the fan module 130, at least one processor 140, the manipulation interface 150, a communication interface 160, a speaker 170, a microphone 180, and a display 190. A description overlapping with FIG. 2 will be omitted.

The memory 110 may be implemented as an internal memory such as a read-only memory (ROM) (for example, electrically erasable programmable read-only memory (EEPROM)) and a random-access memory (RAM) or a memory separate from the processor 140. In this case, the memory 110 may be implemented as a memory embedded in the electronic mask 100 for a data storage use, or a memory type detachable from the electronic mask 100. For example, the data for driving the electronic mask 100 may be stored in a memory embedded in the electronic mask 100, and the data for an expansion of the electronic mask 100 may be stored in a memory detachable from the electronic mask 100.

A memory embedded in the electronic mask 100 may be implemented as at least one of a volatile memory such as a dynamic random access memory (DRAM), a static random access memory (SRAM), a synchronous dynamic random access memory (SDRAM), or a non-volatile memory (for example, one time programmable ROM (OTPROM), programmable ROM (PROM), erasable and programmable ROM (EPROM), electrically erasable and programmable ROM (EEPROM), mask ROM, flash ROM, a flash memory (for example, NAND flash or NOR flash), a hard disk drive (HDD), and a solid state drive (SSD). In the case of a memory detachably mounted to the electronic mask 100, the memory may be implemented as a memory card (for example, a compact flash (CF), secure digital (SD), micro secure digital (micro-SD), mini secure digital (mini-SD), extreme digital (xD), multi-media card (MMC), etc.), an external memory (for example, a universal serial bus (USB) memory) connectable to the USB port, or the like.

The manipulation interface 150 may be implemented as a button, a touch pad, a mouse, and a keyboard, or may be implemented as a touch screen which may perform the display function and a manipulation input function as well. Here, the button may be various types of buttons such as a mechanical button, a touch pad, a wheel, or the like formed on an arbitrary region such as a front portion, a side portion, a back portion, or the like, of an outer part of the main body of the electronic mask 100.

The communication interface 160 perform communication with various types of external devices according to various types of communication methods. The communication interface 160 may include a wireless communication module or a wired communication module. Here, each communication module may be implemented as at least one hardware chip.

The wireless communication module may be a module to wirelessly communicate with an external device. For example, the wireless communication module may include a Wi-Fi module, a Bluetooth module, an infrared ray communication module, or other communication modules.

The Wi-Fi module and the Bluetooth module may perform communication using Wi-Fi method and Bluetooth method, respectively. When using the Wi-Fi module or the Bluetooth module, various connection information such as a service set identifier (SSID) and a session key may be transmitted and received first, and communication information may be transmitted after communication connection.

The infrared ray communication module performs communication according to infrared data association (IrDA) technology that transmits data wireless to local area using infrared ray between visible rays and millimeter waves.

Other communication modules may include at least one communication chip performing communication according to various communication standards such as Zigbee, 3rd generation (3G), 3rd generation partnership project (3GPP), long term evolution (LTE), LTE advanced (LTE-A), 4th generation (4G), 5th generation (5G), or the like.

For example, the wired communication module may include at least one of a local area network (LAN) module, Ethernet module, a pair cable, a coaxial cable, an optical cable, and a ultra wide-band (UWB) module.

The speaker 170 may be a configuration to output various audio data, various alarm sounds, a voice message, or the like.

The microphone 180 may receive user voice in an active state. For example, the microphone 180 may be integrally formed as an integral unit on an upper side, a front side direction, a side direction, or the like of the electronic mask 100. The microphone 180 may include various configurations such as a microphone for collecting user voice in an analog format, an amplifier circuit for amplifying the collected user voice, an audio-to-digital (A/D) conversion circuit for sampling the amplified user voice to convert into a digital signal, a filter circuitry for removing a noise element from the converted digital signal, or the like.

The display 190 may be implemented as various types of a display such as a liquid crystal display (LCD), an organic light emitting diode (OLED) display, a plasma display panel (PDP), or the like. In the display 190, a driving circuit and a backlight unit, which may be implemented in the form of an a-si TFT, a low temperature poly silicon (LTPS) TFT, an organic TFT (OTFT) may be included as well. The display 190 may be implemented as a touch screen coupled with a touch sensor, a flexible display, a third-dimensional display (3D display), or the like. The display 190 according to an embodiment may include not only a display panel for outputting an image but also a bezel for housing a display panel. In particular, the bezel according to an embodiment may include a touch sensor to sense a user interaction.

The display 190 may include light emitting diodes (LED). The electronic mask 100 may control so that an LED of a preset color emits light according to a control command.

In certain embodiments, the electronic mask 100 may include a power supply. The power supply may supply power to various hardware configurations included in the electronic mask 100.

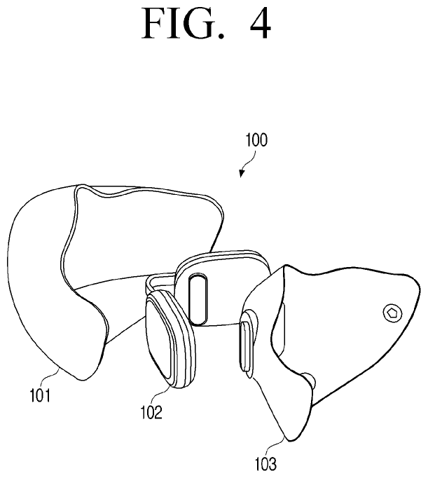

FIG. 4 is a diagram illustrating a structure of the electronic mask 100 according to various embodiments.

Referring to FIG. 4, the electronic mask 100 may include at least one of an outer skin 101, a main body 102, and an inner skin 103.

The outer skin 101 may be disposed to surround the main body 102. The outer skin 101 may be disposed at an outermost side of the electronic mask 100.

The main body 102 may include a fan module 130.

The inner skin 103 may be disposed at an innermost side of the electronic mask 100. The inner skin 103 may be a portion in contact with the facial skin of a user. The inner skin 103 may include a basic filter.

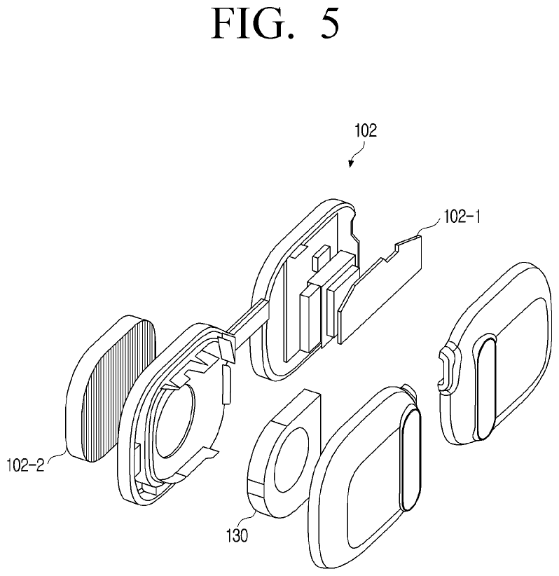

FIG. 5 is a diagram illustrating a structure of the electronic mask 100 according to various embodiments.

Referring to FIG. 5, the main body 102 may include at least one of the fan module 130, a printed board assembly (PBA) 102-1, and a filter 102-2.

The fan module 130 may include at least one of a fan and a motor. According to various embodiments, the fan module 130 may include at least one of an inhalation module and an exhalation module. The inhalation module includes an inhalation fan and an inhalation motor, and the exhalation module may include an exhalation fan and an exhalation motor.

The PBA 102-1 may be circuit substrate including at least one of the pressure sensor 120 and the microphone 180.

The filter 102-2 may be a member for filtering particles of a specific size or less. According to various embodiments, the electronic mask 100 may supply power to the filter 102-2 in order to perform a function of the filter 102-2. The electronic mask 100 may control on or off of the filter 102-2 according to a control command.

FIG. 6 is a flowchart illustrating an operation of storing default atmospheric pressure information.

Referring to FIG. 6, the electronic mask 100 may obtain a control command for obtaining default atmospheric pressure information in operation S605. The default atmospheric pressure information may include information related to a general breathing pattern of a user wearing the electronic mask 100. There may be a unique breathing pattern for each person. The breathing pattern may include information about an atmospheric pressure of inhalation, an atmospheric pressure of exhalation, a breathing time, and the like.

The electronic mask 100 may obtain atmospheric pressure information for a preset time in operation S610. The electronic mask 100 may obtain a minimum default atmospheric pressure and a maximum default atmospheric pressure based on the obtained atmospheric pressure information in operation S615. The minimum default atmospheric pressure may indicate a minimum atmospheric pressure among atmospheric pressure obtained for a preset time. The maximum default atmospheric pressure may indicate a maximum atmospheric pressure among atmospheric pressure obtained for a preset time.

The electronic mask 100 may store default atmospheric pressure information including a minimum default atmospheric pressure and a maximum default atmospheric pressure in operation S620. An atmospheric pressure range between the maximum default atmospheric pressure and the minimum default atmospheric pressure may be an atmospheric pressure corresponding to a general breathing pattern of the user. Therefore, the electronic mask 100 may store default atmospheric pressure information based on atmospheric pressure information obtained for a preset time.

According to various embodiments, the electronic mask 100 may obtain preset atmospheric pressure information and transmit the obtained atmospheric pressure information to the terminal device 200. The terminal device 200 may obtain default atmospheric pressure information based on the received atmospheric pressure information. The terminal device 200 may transmit the obtained default atmospheric pressure information to the electronic mask 100. The electronic mask 100 may store default atmospheric pressure information received from the terminal device 200.

FIG. 7 is a diagram illustrating an operation of storing default atmospheric pressure information.

A table 710 of FIG. 7 may indicate atmospheric pressure information obtained during a preset time.

The electronic mask 100 may obtain default atmospheric pressure information based on the atmospheric pressure information obtained during a preset time. It is assumed that the preset time is greater than tn6.

The electronic mask 100 may obtain a minimum default atmospheric pressure (pn1) of atmospheric pressure information obtained for a preset time. In addition, the electronic mask 100 may obtain a maximum default atmospheric pressure (pn2) of atmospheric pressure information obtained for a preset time. The electronic mask 100 may obtain default atmospheric pressure information including a minimum default atmospheric pressure (pn1) and a maximum default atmospheric pressure (pn2).

The electronic mask 100 may obtain an extreme point, at least one of an extreme maximum point and an extreme minimum point, based on atmospheric pressure information obtained for a preset time. The extreme maximum point may refer to a point at which atmospheric pressure rises and then immediately descends. The extreme minimum point may mean a point at which atmospheric pressure descends and then immediately rises. The extreme point may mean a point where the slope of the atmospheric pressure becomes zero (0). The extreme point may mean a point where the slope of a tangent line is 0 in a function for atmospheric pressure.

The points in time at which the extreme maximum points are obtained may be written as th1, th2, and th3.

The points in time at which the extreme minimum points are obtained may be written as tl1, tl2, and tl3.

The electronic mask 100 may obtain the default breathing time based on the time at which the extreme point is obtained.

According to various embodiments, the electronic mask 100 may obtain a time interval of an extreme maximum point as a default breathing time. For example, the electronic mask 100 may obtain a default breathing time of td1 [=th2−th1] or th3 [=th3−th2]. According to various embodiments, when a plurality of time intervals are obtained, the electronic mask 100 may obtain an average value [(td1+th3)/2] of a plurality of time intervals as a default breathing time.

According to various embodiments, the electronic mask 100 may obtain a time interval of an extreme minimum point as a default breathing time. For example, the electronic mask 100 may obtain td2=tl2−tl1 or td4=tl3−tl2 as a default breathing time. According to various embodiments, when a plurality of time intervals are obtained, the electronic mask 100 may obtain an average value [(td2+td4)/2] of a plurality of time intervals as a default breathing time.

According to various embodiments, the electronic mask 100 may obtain an average of all values [td1+td2+td3+td4)/4] of time intervals as a default breathing time.

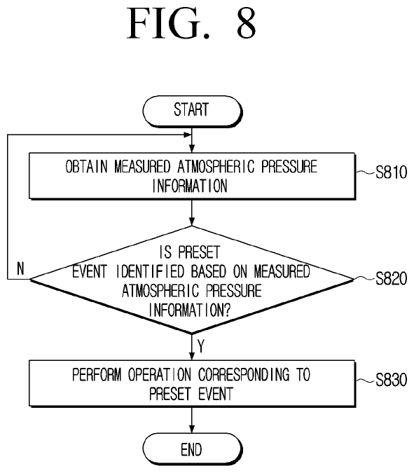

FIG. 8 is a flowchart illustrating an operation of identifying a preset event by using measured atmospheric pressure information.

Referring to FIG. 8, the electronic mask 100 may obtain measured atmospheric pressure information in operation S810. The electronic mask 100 may sense an atmospheric pressure of the inner part of the electronic mask in real time. Data related to the sensed atmospheric pressure may be included in the measured atmospheric pressure information.

The electronic mask 100 may identify a preset event based on measured atmospheric pressure information in operation S820. The preset event may mean an event in which a specific breathing pattern is identified or generated.

When a preset event is not identified in operation S820-N, the electronic mask 100 may repeat operations S810 to S820.

When a preset event is identified in operation S820-Y, the electronic mask 100 may perform an operation corresponding to a preset event in operation S830. The operation corresponding to the preset event may refer to an operation of performing a specific mode or an operation of controlling the electronic mask 100 in a specific state. A detailed description related to the same is described in FIG. 27.

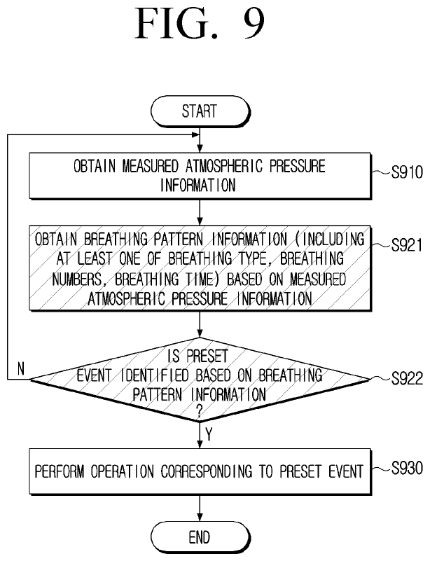

FIG. 9 is a flowchart illustrating an operation of identifying breathing pattern information based on measured atmospheric pressure information.

The operations S910 and S930 of FIG. 9 may correspond to operations S810 and S830 of FIG. 8. Therefore, a duplicate description will be omitted.

After the measured atmospheric pressure information is obtained, the electronic mask 100 may obtain breathing pattern information based on the measured atmospheric pressure information in operation S921. The breathing pattern information may include at least one of a breathing type, a breathing number, and a breathing time.

The breathing type may include exhalation or inhalation. Specifically, exhalation may include short exhalation or long exhalation. In addition, inhalation may include short inhalation or long inhalation. A description related to exhalation is described in FIGS. 12-15. A description related to inhalation is described in FIGS. 16-19.

The breathing number may mean an amount of times a specific breathing type is identified.

The breathing time may mean a time during which a specific type of breathing is performed.

The electronic mask 100 may identify a preset event based on breathing pattern information in operation S922. When a preset event is not identified in operation S922-N, the electronic mask 100 may repeat operations S910 to S922.

When a preset event is identified in operation S922-Y, the electronic mask 100 may perform operation S930.

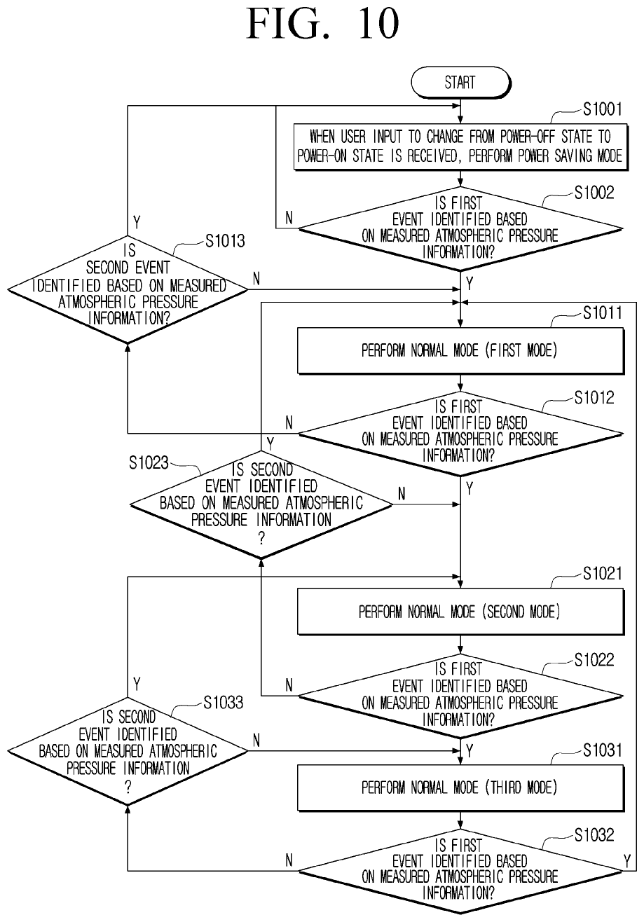

FIG. 10 is a flowchart illustrating a plurality of normal modes.

Referring to FIG. 10, when a user input for changing from a power-off state to a power-on state is received, the electronic mask 100 may perform a power saving mode in operation S1001. The power saving mode may be a mode in which the fan motor is not driven while sensing atmospheric pressure through the pressure sensor.

While the power saving mode is performed, the electronic mask 100 may obtain measured atmospheric pressure information.

The electronic mask 100 may determine whether the first event is identified based on the measure atmospheric pressure information in operation S1002.

When the first event is not identified during when the power saving mode is performed in operation S1002-N, the electronic mask 100 may repeat operations S1001 to S1002.

When the first event is identified during when the power saving mode is performed in operation S1002-Y, the electronic mask 100 may perform a first mode among normal modes in operation S1011. The first mode may be a mode of driving the fan mode with the first intensity.

The electronic mask 100 may determine whether the first event is identified based on the measured atmospheric pressure information obtained while the first mode is performed in operation S1012.

When the first event is not identified during when the first mode is performed in operation S1012-N, the electronic mask 100 may determine whether the second event is identified based on the measured atmospheric pressure information in operation S1013. The first event and the second event may be different events.

If a second event is not identified while the first mode is performed in operation S1013-N, the electronic mask 100 may continue to perform the first mode. When a second event is identified while the first mode is performed in operation S1013-Y, the electronic mask 100 may perform a power saving mode in operation S1001.

When the first event is identified while the first mode is performed in operation S1012-Y, the electronic mask 100 may perform a second mode among the normal modes in operation S1021.

The electronic mask 100 may determine whether the first event is identified based on the obtained measured atmospheric pressure information during which the second mode is performed in operation S1022.

When the first event is not identified during when the second mode is performed in operation S1022-N, the electronic mask 100 may determine whether the second event is identified based on the measured atmospheric pressure information in operation S1023. The first event and the second event may be different events.

If a second event is not identified while the second mode is performed in operation S1023-N, the electronic mask 100 may continue to perform a second mode. When the second event is identified while the second mode is performed in operation S1023-Y, the electronic mask 100 may perform the first mode in operation S1011.

When the first event is identified while the second mode is performed in operation S1022-Y, the electronic mask 100 may perform a third mode among the normal modes in operation S1031.

The electronic mask 100 may determine whether the first event is identified based on the obtained measured atmospheric pressure information while the third mode is performed in operation S1032.

If the first event is not identified while the third mode is performed in operation S1032-N, the electronic mask 100 may determine whether the second event is identified based on the measured atmospheric pressure information in operation S1033. The first event and the second event may be different events.

If a second event is not identified while the third mode is performed in operation S1033-N, the electronic mask 100 may continue to perform a third mode. When a second event is identified while the third mode is performed in operation S1033-Y, the electronic mask 100 may perform a second mode in operation S1021.

When the first event is identified while the third mode is performed in operation S1032-Y, the electronic mask 100 may perform the first mode among the normal modes in operation S1011.

According to various embodiments, when the first event is identified during when the third mode is performed in operation 1032-Y, the electronic mask 100 may keep performing the third mode.

According to various embodiments, there may be a fourth mode instead of the third mode. When the first event is identified while the third mode is performed in operation S1032-Y, the electronic mask 100 may perform the fourth mode.

FIG. 11 is a diagram illustrating a state of the electronic mask 100.

The table 1110 of FIG. 11 may indicate whether the function of the electronic mask 100 is performed according to a state or mode of the electronic mask 100.

In the power-off state, the electronic mask 100 may not supply power. The electronic mask 100 may not perform a function related to a pressure sensor, a fan motor, a general filter, and an additional filter.

In the power saving mode or standby power mode, the electronic mask 100 may supply power. In addition, the electronic mask 100 may sense an atmospheric pressure by using a pressure sensor. In addition, the electronic mask 100 may not perform functions for a fan motor, a general filter, and an additional filter.

In a general mode (e.g., a first mode, a second mode, and a third mode), the electronic mask 100 may supply power. In addition, the electronic mask 100 may sense an atmospheric pressure by using a pressure sensor. In addition, the electronic mask 100 may control a fan motor and a general filter. The electronic mask 100 may rotate the fan by driving the fan motor. In addition, the electronic mask 100 may perform a filter function by supplying power to a general filter.

In a high performance mode, the electronic mask 100 may supply power. In addition, the electronic mask 100 may sense an atmospheric pressure by using a pressure sensor. In addition, the electronic mask 100 may perform a function related to a fan motor, a general filter, and an additional filter. The electronic mask 100 may supply power to an additional filter to perform an additional filter function. According to various embodiments, a high performance mode may be omitted.

A table 1120 indicates a plurality of modes included in a normal mode. The normal mode may include a first mode, a second mode, and a third mode. The first mode may be a mode in which the fan motor is driven at a first intensity M1, the second mode may be a mode in which the fan motor is driven at a second intensity M2, and the third mode may be a mode in which the fan motor is driven at a third intensity M3. The second intensity M2 may be a value greater than the first intensity M1, and the third intensity M3 may be a value greater than the second intensity M2.

FIG. 12 is a diagram illustrating an embodiment in which a short exhalation event is identified once.

The table 1210 of FIG. 12 may indicate the measured atmospheric pressure information in which the short exhalation event is identified once during a preset time. In order to identify the exhalation event, there may be event atmospheric pressure (exhalation event atmospheric pressure information). The event atmospheric pressure (exhalation event atmospheric pressure information) corresponding to the exhalation event may include exhalation event atmospheric pressure Po2. The exhalation event atmospheric pressure (Po2) may mean a threshold atmospheric pressure to identify an exhalation event. The electronic mask 100 may, when the atmospheric pressure which is greater than or equal to the exhalation event atmospheric pressure Po2 is measured, identify that the exhalation event is identified.

The exhalation event may include a short exhalation event and a long exhalation event. In order to divide the short exhalation event and the long exhalation event, the electronic mask 100 may use the exhalation event time in which the atmospheric pressure that is greater than or equal to the event atmospheric pressure Po2.

The exhalation event may indicate the time (t2−t1) at which the atmospheric pressure that is greater than or equal to the event atmospheric pressure Po2 is measured.

The electronic mask 100 may obtain the time point (t1, t2) at which the event atmospheric pressure Po2 is identified. T1 may be a time point at which the atmospheric pressure less than the event atmospheric pressure Po2 is being identified and then the event atmospheric pressure Po2 is identified. T2 may be a time point at which atmospheric pressure greater than the event atmospheric pressure Po2 is identified and then the event atmospheric pressure Po2 is identified.

The electronic mask 100 may obtain, as the exhalation event time, time information (t2−t1) at which the atmospheric pressure greater than or equal to event atmospheric pressure Po2 is identified.

The electronic mask 100 may distinguish a short exhalation event and a long exhalation event by comparing an exhalation event time and a threshold time. Specifically, when an exhalation event time is equal to or greater than a threshold time, the electronic mask 100 may determine that a long exhalation event has been identified. If the exhalation event time is less than a threshold time, the electronic mask 100 may determine that a short exhalation event has been identified.

In the embodiment of FIG. 12, the exhalation event time (t2−t1) is less than a threshold time and the electronic mask 100 may identify that the exhalation event is a short exhalation event.

When the exhalation event is identified, the electronic mask 100 may identify the number of events and breathing time based on the extreme maximum point.

When the exhalation event is identified, the electronic mask 100 may identify the number of obtaining extreme maximum points in the exhalation event time range from t1 to t2 as the number of events.

When an exhalation event is identified, the electronic mask 100 may identify the extreme maximum points 1211 and 1212. In addition, the electronic mask 100 may identify the number of extreme maximum values in the exhalation event time range from t1 to t2. The electronic mask 100 may identify the number of identified extreme maximum values as an event occurrence number.

In the embodiment of FIG. 12, the number of extreme maximum points 1211 identified in the exhalation event time range from t1 to t2 may be one (1). Therefore, the electronic mask 100 may identify the number of events as once.

According to various embodiments, the electronic mask 100 may determine whether the points in time th1 and th2 at which the extreme maximum points 1211 and 1212 are obtained is included in the exhalation event time ranges from t1 to t2. Among the point in time th1 and th2 at which the extreme maximum points 1211 and 1212 are obtained, the number (1) included in the exhalation event time ranges from t1 to t2 may be identified as the numbers of events.

According to various embodiments, the electronic mask 100 may identify the number (1) of the time points at which the extreme maximum value corresponding to the time points th1 and th2 at which the extreme maximum points 1211 and 1212 are obtained is greater than or equal to the atmospheric pressure Po2 as the number of events.

When the exhalation event is identified, the electronic mask 100 may identify the difference of time points when the extreme maximum point is obtained as the breathing time.

The electronic mask 100 may identify points in time th1 and th2 at which the extreme maximum points 1211 and 1212 are obtained. In addition, the electronic mask 100 may obtain a difference (th2−th1) of a time point at which the extreme maximum point is obtained. The electronic mask 100 may obtain a difference (th2−th1) of a time point as a breathing time.

According to various embodiments, the electronic mask 100 may determine whether the breathing time (th2−th1) is the same as the default breathing time. If the breathing time (th2−th1) is equal to the default breathing time, the electronic mask 100 may identify that a preset event has not occurred.

Referring to table 1220, the electronic mask 100 may obtain breathing pattern information including at least one of an event time (t2−t1), an event type (e.g., a short exhalation), the number of events (one), and a breathing time (th2−th1). The electronic mask 100 may store obtained breathing pattern information.

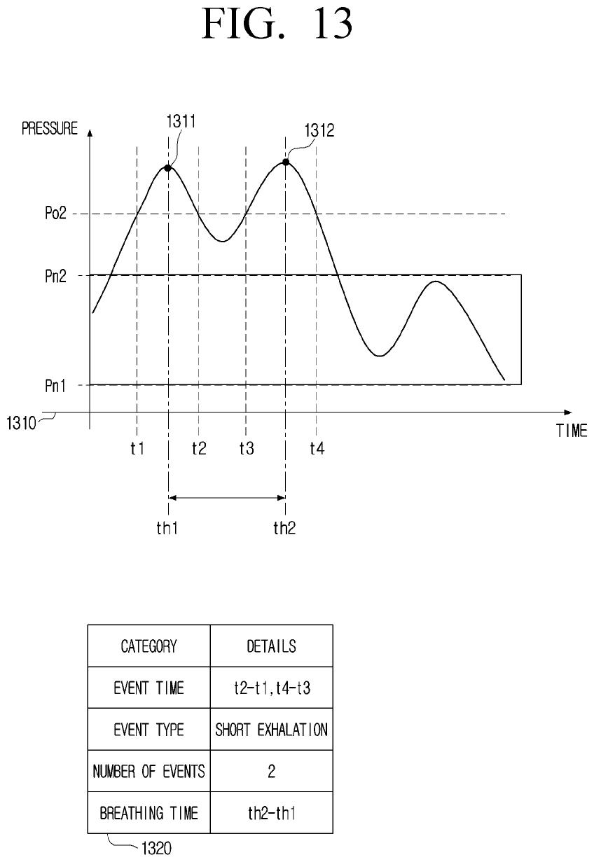

FIG. 13 is a diagram illustrating an embodiment in which a short exhalation event is identified twice.

The table 1310 of FIG. 13 may indicate the measured atmospheric pressure information that short exhalation events are identified by two times during a preset time. In the embodiment of FIG. 13, an operation illustrated in the embodiment of FIG. 12 is applicable. Therefore, a duplicate description will be omitted.

The exhalation event time may indicate times (t2−t1 and t4−t3) at which atmospheric pressure, which is greater than or equal to an event atmospheric pressure Po2, is measured. The electronic mask 100 may obtain time information (t2−t1 and t4−t3) in which atmospheric pressure, which is greater than or equal to the event atmospheric pressure Po2, is identified as an exhalation event time.

In the embodiment of FIG. 13, it is assumed that the exhalation event times (t2−t1 and t4−t3) is less than a threshold time. The electronic mask 100 may identify that the exhalation event is a short exhalation event.

When the exhalation event is identified, the electronic mask 100 may identify the event times and breathing time based on the extreme maximum point.

When an exhalation event is identified, the electronic mask 100 may identify the number of obtained extreme maximum points in the exhalation event time range from t1 to t2 and from t3 to t4 as the number of events. When an exhalation event is identified, the electronic mask 100 may identify the extreme maximum points 1311 and 1312. In addition, the electronic mask 100 may identify the number of extreme maximum values in the exhalation event time ranges from t1 to t2 and from t3 to t4. The electronic mask 100 may identify the number of identified extreme maximum values as an event occurrence number.

In the embodiment of FIG. 13, the number of extreme maximum points 1311 and 1312 identified in the exhalation event time range from t1 to t2 and from t3 to t4 may be two. Therefore, the electronic mask 100 may identify the number of events as twice.

According to various embodiments, the electronic mask 100 may determine whether the time points th1 and th2 at which the extreme maximum points 1311 and 1312 are obtained are included in the exhalation event time ranges from t1 to t2 and from t3 to t4. The number (2) included in the exhalation event time ranges from t1 to t2 and from t3 to t4 among the time points th1 and th2 at which the extreme maximum points 1311 and 1312 are obtained may be identified as the number of events.

According to various embodiments, the electronic mask 100 may identify the number (2) of times the extreme maximum value corresponding to the time points th1 and th2 at which the extreme maximum points 1311 and 1312 are obtained is equal to or greater than the event atmospheric pressure Po2 as the number of events.

When the exhalation event is identified, the electronic mask 100 may identify the difference of the time points at which the extreme maximum point is obtained as the breathing time.

The electronic mask 100 may identify the time points th1 and th2 at which the extreme maximum points 1311 and 1312 are obtained. In addition, the electronic mask 100 may obtain the difference (th2−th1) of time points at which the extreme maximum point is obtained. The electronic mask 100 may obtain the difference (th2−th1) of time points as the breathing time.

According to various embodiments, the electronic mask 100 may determine whether the breathing time (th2−th1) is equal to the default breathing time. If the breathing time (th2−th1) is equal to the default breathing time, the electronic mask 100 may identify that a preset event has not occurred.

Referring to table 1320, the electronic mask 100 may obtain breathing pattern information including at least one of an event time (t2−t1 and t4−t3), an event type (e.g., a short exhalation), the number of events (twice), and a breathing time (th2−th1). The electronic mask 100 may store obtained breathing pattern information.

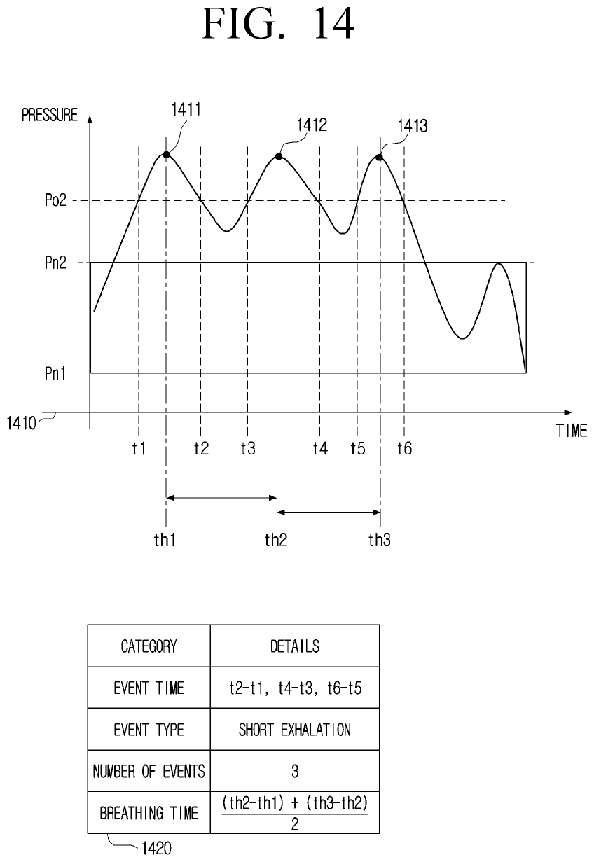

FIG. 14 is a diagram illustrating an embodiment in which a short exhalation event is identified thrice.

The table 1410 of FIG. 14 may indicate that the measured atmospheric pressure information that short exhalation events are identified by three times during a preset time. In the embodiment of FIG. 14, an operation illustrated in the embodiment of FIG. 12 is applicable. Therefore, a duplicate description will be omitted.

The exhalation event time may indicate times (t2−t1, t4−t3, t6−t5) at which atmospheric pressure, which is at least an event atmospheric pressure Po2, is measured. The electronic mask 100 may obtain time information (t2−t1, t4−t3, t6−t5) in which atmospheric pressure, which is greater than or equal to the event atmospheric pressure Po2, is identified as an exhalation event time.

In the embodiment of FIG. 14, it is assumed that the exhalation event time (t2−t1, t4−t3, t6−t5) is less than a threshold time. The electronic mask 100 may identify that the exhalation event is a short exhalation event.

When the exhalation event is identified, the electronic mask 100 may identify the event times and breathing time based on the extreme maximum point.

When the exhalation event is identified, the electronic mask 100 may identify the number of extreme maximum points obtained in the exhalation event time range from t1 to t2, from t3 to t4, and from t5 to t6 as the number of events. When an exhalation event is identified, the electronic mask 100 can identify the extreme maximum points 1411, 1412, and 1413. In addition, the electronic mask 100 may identify the number of extreme maximum values in the exhalation event time range from t1 to t2, from t3 to t4, and from t5 to t6. The electronic mask 100 may identify the number of identified extreme maximum values as an event occurrence number.

In the embodiment of FIG. 14, the number of extreme maximum points 1411, 1412, and 1413 identified in the exhalation event time range from t1 to t2, from t3 to t4, and from t5 to t6 may be three. Therefore, the electronic mask 100 may identify the number of events as thrice.

According to various embodiments, the electronic mask 100 may determine whether the time points th1, th2, and th3 at which the extreme maximum points 1411, 1412, and 1413 are obtained are included in the exhalation event time ranges from t1 to t2, from t3 to t4, and from t5 to t6. The number (3) included in the exhalation event time ranges (t1 to t2, t3 to t4, and t5 to t6) among the time points th1, th2, and th3 at which the extreme maximum points 1411, 1412, and 1413 are obtained may be identified as the number of events.

According to various embodiments, the electronic mask 100 may identify the number (3) of the time points at which the extreme maximum value corresponding to the time points th1, th2, and th3 at which the extreme maximum points 1411, 1412, and 1413 are obtained is equal to or greater than the event atmospheric pressure Po2 as the number of events.

When the exhalation event is identified, the electronic mask 100 may identify the difference of the time points at which the extreme maximum point is obtained as the breathing time.

The electronic mask 100 may identify the time points th1, th2, and th3 at which the extreme maximum points 1411, 1412, and 1413 are obtained. In addition, the electronic mask 100 may obtain the difference (th2−th1 and th3−th2) of time points at which the extreme maximum point is obtained. When the differences of time points are plural, the electronic mask 100 may obtain an average [{(th2−th1)+(th3−th2)}/2] of a plurality of difference values as the breathing time.

According to various embodiments, the electronic mask 100 may determine whether the breathing time [{(th2−th1)+(th3−th2)}/2] is the same as the default breathing time. If the breathing time [{(th2−th1)+(th3−th2)}/2] is the same as the default breathing time, the electronic mask 100 may identify that a preset event has not occurred.

Referring to table 1420, the electronic mask 100 may obtain breathing pattern information including at least one of the event time (t2−t1, t4−t3, and t6−t5), event type (a short exhalation), number of events (thrice), and breathing time [{(th2−th1)+(th3−th2)}/2]. The electronic mask 100 may store obtained breathing pattern information.

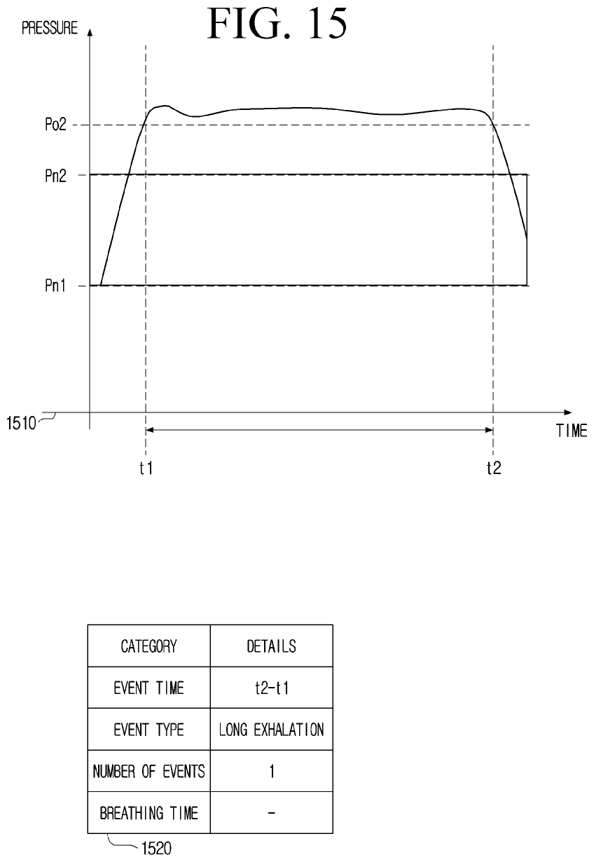

FIG. 15 is a diagram illustrating an embodiment in which a long exhalation event is identified.

The table 1510 of FIG. 15 may indicate that the measured atmospheric pressure information that long exhalation event is identified by once during a preset time. In the embodiment of FIG. 15, an operation illustrated in the embodiment of FIG. 12 is applicable. Therefore, a duplicate description will be omitted.

The exhalation event time may indicate time (t2−t1) at which atmospheric pressure, which is greater than or equal to an event atmospheric pressure Po2, is measured. The electronic mask 100 may obtain time information (t2−t1) at which atmospheric pressure, which is greater than or equal to the event atmospheric pressure Po2, is identified as an exhalation event time.

In the embodiment of FIG. 15, it is assumed that the exhalation event time (t2−t1) is greater than a threshold time. The electronic mask 100 may identify that the exhalation event is a long exhalation event.

When the long exhalation event is identified, the electronic mask 100 may identify the number of events. In the embodiment of FIG. 15, the exhalation event may be once.

When a long exhalation event is identified, the electronic mask 100 may not separately store the breathing time. According to various embodiments, the electronic mask 100 may obtain a breathing time as an event time.

Referring to table 1520, the electronic mask 100 may obtain breathing pattern information including at least one of an event time (t2−t1), an event type (a long exhalation), the number of events (once), and a breathing time [−]. The electronic mask 100 may store obtained breathing pattern information.

FIG. 16 is a diagram illustrating an embodiment in which a short inhalation event is identified once.

The table 1610 of FIG. 16 may indicate that the measured atmospheric pressure information that short inhalation event is identified by once during a preset time. In order to identify an inhalation event, atmospheric pressure information (inhalation event atmospheric pressure information) may be present. The event atmospheric pressure information (inhalation event atmospheric pressure information) corresponding to the inhalation event may include inhalation event atmospheric pressure Po1. The inhalation event atmospheric pressure Po1 may indicate a threshold atmospheric pressure to identify an inhalation event. The electronic mask 100 may, if the atmospheric pressure less than or equal to inhalation event atmospheric pressure Po1 is measured, determine that the inhalation event is identified.

The inhalation event may include a short inhalation event and a long inhalation event. In order to divide a short inhalation event and a long inhalation event, the electronic mask 100 may use an inhalation event time at which the atmospheric pressure less than or equal to the event atmospheric pressure Po1 is measured.

The inhalation event time may indicate the time (t2−t1) that the atmospheric pressure that is less than or equal to the event atmospheric pressure Po1 is measured.

The electronic mask 100 may obtain the time points (t1 and t2) at which the event atmospheric pressure Po1 is identified. The t1 may be a time point at which atmospheric pressure higher than the event atmospheric pressure Po1 is identified and then the event atmospheric pressure Po1 is identified. The t2 may be a time point at which the atmospheric pressure lower than the event atmospheric pressure Po1 is identified and then the event atmospheric pressure Po1 is identified.

The electronic mask 100 may identify the time information (t2−t1) at which the atmospheric pressure less than or equal to the event atmospheric pressure Po1 is identified as the inhalation event time.

The electronic mask 100 may divide the short inhalation event and the long inhalation event by comparing the inhalation event time and the threshold time. To be specific, when the inhalation event time is greater than or equal to a threshold time, the electronic mask 100 may determine that the long inhalation event is identified. When the inhalation event time is less than a threshold time, the electronic mask 100 may determine that a short inhalation event is identified.

In the embodiment of FIG. 16, it is assumed that the inhalation event time (t2−t1) is smaller than a threshold time. The electronic mask 100 may identify that the inhalation event is a short inhalation event.

When the inhalation event is identified, the electronic mask 100 may identify the number of events and breathing time based on the extreme minimum point.

When the inhalation event is identified, the electronic mask 100 may identify the number of obtaining the extreme minimum point in the inhalation event time range (t1 to t2) as the number of events.

When the inhalation event is identified, the electronic mask 100 may identify the extreme minimum points 1611 and 1612. In addition, the electronic mask 100 may identify the number of extreme minimum values in the inhalation event time ranges from t1 to t2. The electronic mask 100 may identify the number of identified extreme minimum values as an event occurrence number.

In the embodiment of FIG. 16, the number of extreme minimum point 1611 identified in the inhalation event time range from t1 to t2 may be one. Therefore, the electronic mask 100 may identify the number of events as once.

According to various embodiments, the electronic mask 100 may determine whether the time points tl1 and tl2 at which the extreme minimum points 1611 and 1612 are obtained are included in the inhalation event time ranges from t1 to t2. The number (1) included in the inhalation event time ranges from t1 to t2 among the time points tl1 and tl2 at which the extreme minimum points 1611 and 1612 are obtained may be identified as the number of events.

According to various embodiments, the electronic mask 100 may identify the number (1) of time point at which the extreme minimum value corresponding to the time point tl1 and tl2 at which the extreme minimum points 1611 and 1612 are obtained is less than or equal to the event atmospheric pressure Po1 as the number of events.

When the inhalation event is identified, the electronic mask 100 may identify the different of time point of obtaining the extreme minimum point as the breathing time.

The electronic mask 100 may identify the time point tl1 and tl2 at which the extreme minimum points 1611 and 1612 are obtained. The electronic mask 100 may obtain the difference (tl2−tl1) of time point of obtaining the extreme minimum point. The electronic mask 100 may obtain the difference (tl2−tl1) of time point as the breathing time.

According to various embodiments, the electronic mask 100 may determine whether the breathing time (tl2−tl1) is equal to the default breathing time. If the breathing time (tl2−tl1) is equal to the default breathing time, the electronic mask 100 may identify that a preset event has not occurred.

Referring to table 1620, the electronic mask 100 may obtain breathing pattern information including at least one of an event time (t2−t1), an event type (a short inhalation), an number of events (once), and a breathing time (tl2−tl1). The electronic mask 100 may store obtained breathing pattern information.

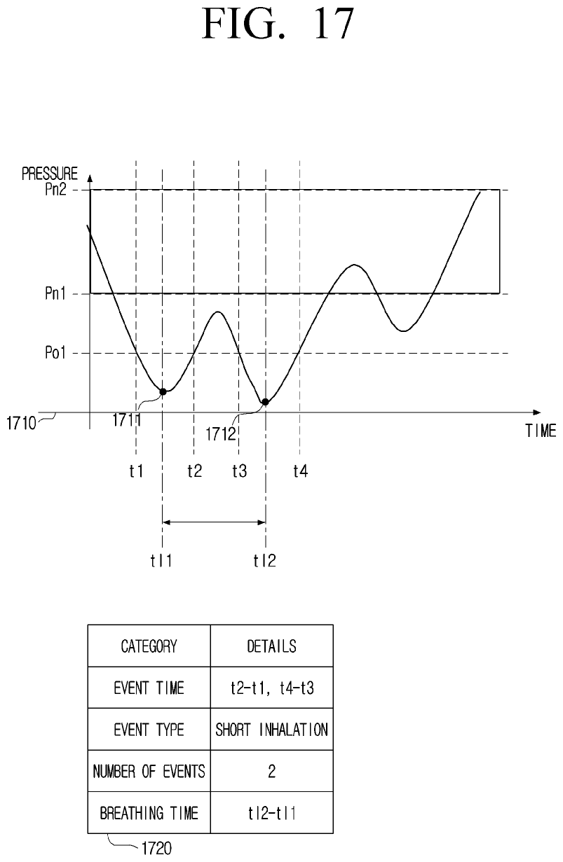

FIG. 17 is a diagram illustrating an embodiment in which a short inhalation event is identified twice.

The table 1710 of FIG. 17 may indicate the measured atmospheric pressure information in which short inhalation event is identified by twice for a preset time. The operation described in FIG. 16 may be applied to the embodiment of FIG. 17. Thus, a duplicate description will be omitted.

The inhalation event time may indicate a time (t2−t1 and t4−t3) at which an atmospheric pressure, which is less than or equal to an event atmospheric pressure Po1, is measured. The electronic mask 100 may obtain time information (t2−t1 and t4−t3) at which atmospheric pressure, which is less than or equal to the event atmospheric pressure Po1, is identified as an inhalation event time.

In the embodiment of FIG. 17, it is assumed that the inhalation event time (t2−t1 and t4−t3) is less than the threshold time. The electronic mask 100 may identify that the inhalation event is a short inhalation event.

When the inhalation event is identified, the electronic mask 100 may identify the number of events and breathing time based on the extreme minimum point.

When an inhalation event is identified, the electronic mask 100 may identify, as the number of events, the number at which an extreme minimum point is obtained in an inhalation event time range from t1 to t2 and from t3 to t4. When an inhalation event is identified, the electronic mask 100 may identify the extreme minimum points 1711 and 1712. In addition, the electronic mask 100 may identify the number of extreme minimum values in the inhalation event time ranges from t1 to t2 and from t3 to t4. The electronic mask 100 may identify the number of identified extreme minimum values as an event occurrence number.

In the embodiment of FIG. 17, the number of extreme minimum points 1711 and 1712 identified in the inhalation event time range from t1 to t2 and from t3 to t4 may be two (2). Therefore, the electronic mask 100 may identify the number of events as twice.

According to various embodiments, the electronic mask 100 may determine whether the time points tl1 and tl2 at which the extreme minimum points 1711 and 1712 are obtained are included in the inhalation event time ranges from t1 to t2 and from t3 to t4. The number (2) included in the inhalation event time ranges from t1 to t2 and from t3 to t4 among the time points tl1 and tl2 at which the extreme minimum points 1711 and 1712 are obtained may be identified as the number of events.

According to various embodiments, the electronic mask 100 may identify the number (2) of the time point at which the extreme minimum value corresponding to the time point tl1 and tl2 at which the extreme minimum points 1711 and 1712 are obtained is less than or equal to the event atmospheric pressure Po1 as the number of events.

When the inhalation event is identified, the electronic mask 100 may identify the difference of time points of obtaining the extreme minimum point as the breathing time.

The electronic mask 100 may identify the time point (tl1, tl2) at which the extreme minimum points 1711 and 1712 are obtained. In addition, the electronic mask 100 may obtain the difference (tl2−tl1) of the time point at which the extreme minimum point is obtained. The electronic mask 100 may obtain the difference (tl2−tl1) of the time point as the breathing time.

According to various embodiments, the electronic mask 100 may determine whether the breathing time (tl2−tl1) is the same as the default breathing time. When the breathing time (tl2−tl1) is the same as the default breathing time, the electronic mask 100 may identify that the preset event has not occurred.

Referring to table 1720, the electronic mask 100 may obtain breathing pattern information including at least one of an event time (t2−t1 and t4−t3), an event type (a short inhalation), the number of events (twice), and a breathing time (tl2−tl1). The electronic mask 100 may store obtained breathing pattern information.

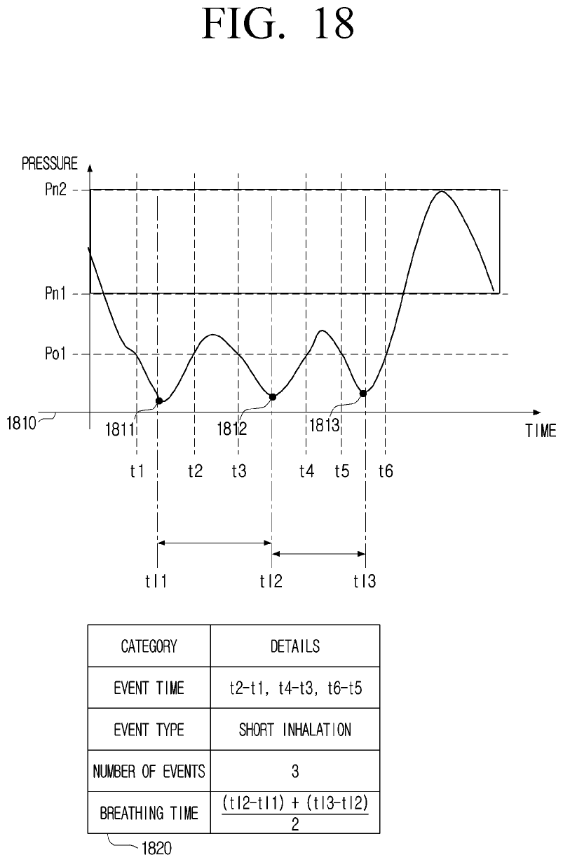

FIG. 18 is a diagram illustrating an embodiment in which a short inhalation event is identified thrice.

The table 1810 of FIG. 18 may indicate that the measured atmospheric pressure information that short inhalation event is identified by thrice during a preset time. In the embodiment of FIG. 18, an operation illustrated in the embodiment of FIG. 12 is applicable. Therefore, a duplicate description will be omitted.

The inhalation event time may indicate time (t2−t1, t4−t3, and t6−t5) at which atmospheric pressure, which is less than or equal to an event atmospheric pressure Po1, is measured. The electronic mask 100 may obtain time information (t2−t1, t4−t3, and t6−t5) at which atmospheric pressure, which is less than or equal to the event atmospheric pressure Po1, is identified as an inhalation event time.

In the embodiment of FIG. 18, it is assumed that the inhalation event time (t2−t1, t4−t3, and t6−t5) is less than a threshold time. The electronic mask 100 may identify that the inhalation event is a short inhalation event.

When the inhalation event is identified, the electronic mask 100 may identify the number of events and breathing time based on the extreme minimum point.

When an inhalation event is identified, the electronic mask 100 may identify, as the number of events, the number at which an extreme minimum point is obtained in an inhalation event time range from t1 to t2, from t3 to t4, and from t5 to t6. When an inhalation event is identified, the electronic mask 100 may identify the extreme minimum points 1811, 1812, and 1813. In addition, the electronic mask 100 may identify the number of extreme minimum values in the inhalation event time ranges from t1 to t2, from t3 to t4, and from t5 to t6. The electronic mask 100 may identify the number of identified extreme minimum values as an event occurrence number.

In the embodiment of FIG. 18, the number of extreme minimum points 1811, 1812, and 1813 identified in the inhalation event time range from t1 to t2, from t3 to t4, and from t5 to t6 may be three (3). Therefore, the electronic mask 100 may identify the number of events as thrice.

According to various embodiments, the electronic mask 100 may determine whether the time points tl1, tl2, and tl3 at which the extreme minimum points 1811, 1812, and 1813 are obtained are included in the inhalation event time ranges from t1 to t2, from t3 to t4, and from t5 to t6. The number (3) included in the inhalation event time ranges from t1 to t2, from t3 to t4, and from t5 to t6 among the time points tl1, tl2, and tl3 at which the extreme minimum points 1811, 1812, and 1813 are obtained may be identified as the number of events.

According to various embodiments, the electronic mask 100 may identify the number (3) of the time points at which the extreme minimum value corresponding to the time points tl1, tl2, and tl3 at which the extreme minimum points 1811, 1812, and 1813 are obtained is equal to or less than the event atmospheric pressure Po1 as the number of events.

When the inhalation event is identified, the electronic mask 100 may identify the difference of the time points at which the extreme minimum point is obtained as the breathing time.

The electronic mask 100 may identify the time points tl1, tl2, and tl3 at which the extreme minimum points 1811, 1812, and 1813 are obtained. In addition, the electronic mask 100 may obtain the difference (tl2−tl1 and tl3−tl2) of time points at which the extreme maximum point is obtained. When the differences of time points are plural, the electronic mask 100 may obtain an average [{(tl2−tl1)+(tl3−tl2)}/2] of a plurality of difference values as the breathing time.

According to various embodiments, the electronic mask 100 may determine whether the breathing time [{(tl2−tl1)+(tl3−tl2)}/2] is the same as the default breathing time. If the breathing time [{(tl2−tl1)+(tl3−tl2)}/2] is the same as the default breathing time, the electronic mask 100 may identify that a preset event has not occurred.

Referring to table 1820, the electronic mask 100 may obtain breathing pattern information including at least one of the event time (t2−t1, t4−t3, and t6−t5), event type (a short inhalation), number of events (thrice), and a breathing time [{(tl2−tl1)+(tl3−tl2)}/2]. The electronic mask 100 may store obtained breathing pattern information.

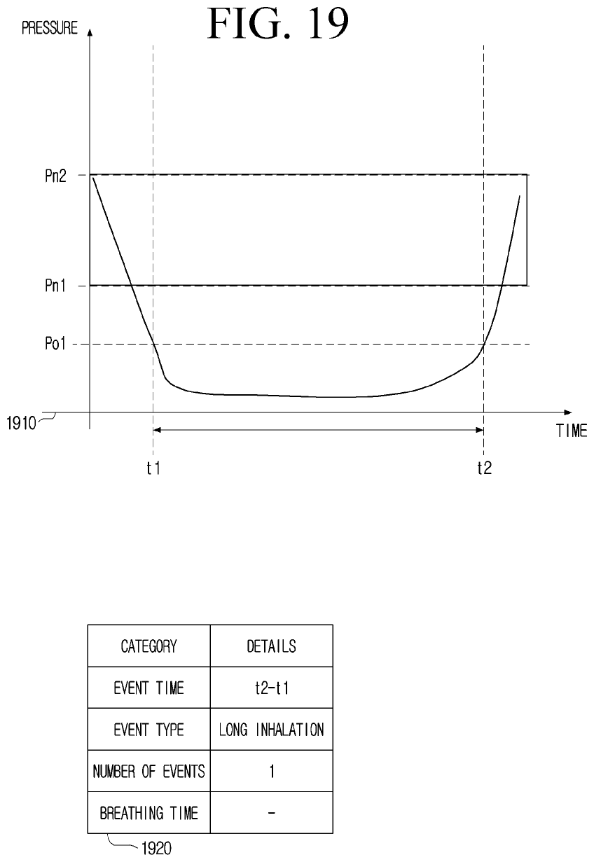

FIG. 19 is a diagram illustrating an embodiment in which a long inhalation event is identified.

The table 1910 of FIG. 19 may indicate that the measured atmospheric pressure information that long inhalation event is identified by once during a preset time. In the embodiment of FIG. 19, an operation illustrated in the embodiment of FIG. 12 is applicable. Therefore, a duplicate description will be omitted.

The inhalation event time may indicate time (t2−t1) at which atmospheric pressure, which is less than or equal to an event atmospheric pressure Po1, is measured. The electronic mask 100 may obtain time information (t2−t1) at which atmospheric pressure, which is less than or equal to the event atmospheric pressure Po1, is identified as an inhalation event time.

In the embodiment of FIG. 19, it is assumed that the inhalation event time (t2−t1) is greater than a threshold time. The electronic mask 100 may identify that the inhalation event is a long inhalation event.

When the long inhalation event is identified, the electronic mask 100 may identify the number of events. In the embodiment of FIG. 15, the inhalation event may be once.

When a long inhalation event is identified, the electronic mask 100 may not separately store the breathing time. According to various embodiments, the electronic mask 100 may obtain a breathing time as an event time.

Referring to table 1920, the electronic mask 100 may obtain breathing pattern information including at least one of an event time (t2−t1), an event type (a long inhalation), the number of events (once), and a breathing time [−]. The electronic mask 100 may store obtained breathing pattern information.

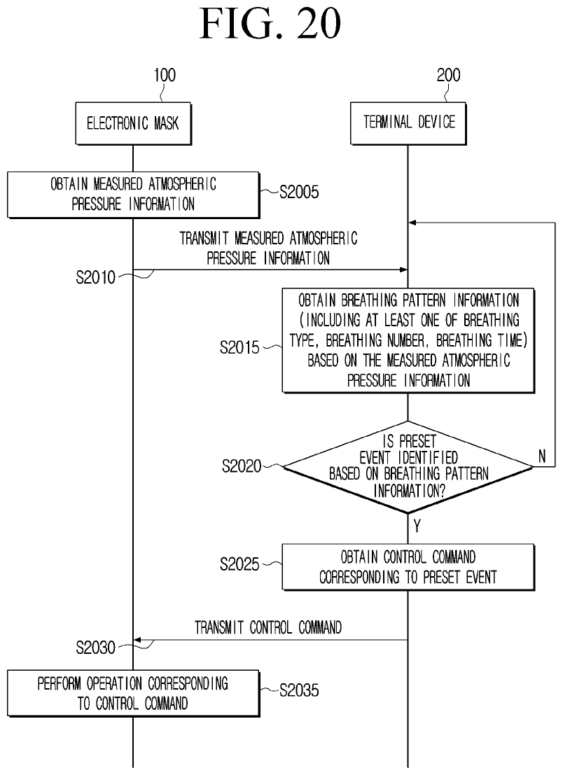

FIG. 20 is a flowchart illustrating an embodiment of identifying a preset event through the terminal device 200.

Referring to FIG. 20, the electronic mask 100 may obtain the measured atmospheric pressure information in operation S2005. The electronic mask 100 may transmit the measured atmospheric pressure information to the terminal device 200 in operation S2010.

The terminal device 200 may receive measured atmospheric pressure information from the electronic mask 100. The terminal device 200 may obtain breathing pattern information based on the measured atmospheric pressure information in operation S2015. The breathing pattern information may include at least one of a breathing type, a breathing rate, and a breathing time. The terminal device 200 may identify a preset event based on the breathing pattern information in operation S2020.

When a preset event is not identified in operation S2020-N, the terminal device 200 may repeatedly perform operations S2015 and S2020.

When a preset event is identified in operation S2020-Y, the terminal device 200 may obtain a control command corresponding to a preset event in operation S2025. The terminal device 200 may transmit an obtained control command to the electronic mask 100 in operation S2030.

The electronic mask 100 may receive a control command from the terminal device 200. The electronic mask 100 may perform an operation (or function) corresponding to the control command in operation S2035.

FIG. 21 is a flowchart illustrating an embodiment of changing a mask setting through the terminal device 200.

Referring to FIG. 21, the terminal device 200 may display a setting screen related to the electronic mask 100 based on a preset event in operation S2105. The terminal device 200 may identify whether a user input for changing a setting is received in operation S2110.

If the user input for changing the setting is not received in operation S210-N, the terminal device 200 may repeat the operation S2110 or terminate the operation S2105.

When a user input for changing setting is received in operation S2110-Y, the terminal device 200 may obtain changed setting information corresponding to a user input in operation S2115. The terminal device 200 may transmit the changed setting information to the electronic mask 100 in operation S2120.

The electronic mask 100 may receive changed setting information from the terminal device 200. The electronic mask 100 may store or apply the changed setting information in operation S2125.

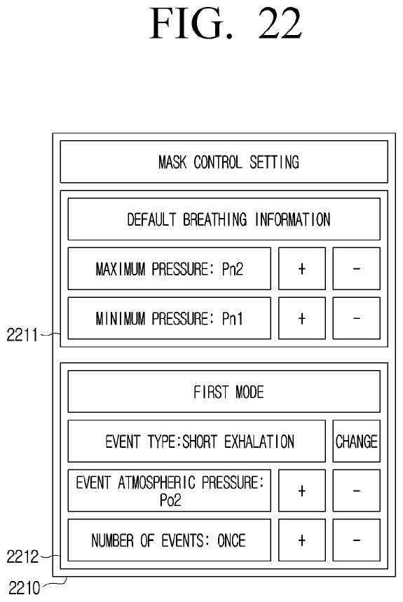

FIG. 22 is a diagram illustrating an operation of controlling a mask setting through an application.

Referring to FIG. 22, the terminal device 200 may display a setting screen 2210 related to the electronic mask 100.

The screen 2210 may include a UI 2211 related to the default atmospheric pressure information or a UI 2212 related to a preset event corresponding to a specific mode.

The UI 2211 related to default atmospheric pressure information may include at least one of a default maximum pressure pn2 and a default minimum pressure pn1. In addition, the UI 2211 related to default atmospheric pressure information may include a guide UI (for example, “+”, “−”) for changing a default maximum pressure pn2 or a default minimum pressure pn1.

The UI 2212 related to the preset event may include at least one of an event type (a short exhalation), an event atmospheric pressure Po2, and an number of events (once) corresponding to a specific mode (first mode). In addition, the UI 2212 related to the preset event may include a guide UI for changing at least one of an event type (a short exhalation), an event atmospheric pressure Po2, and the number of events (once).

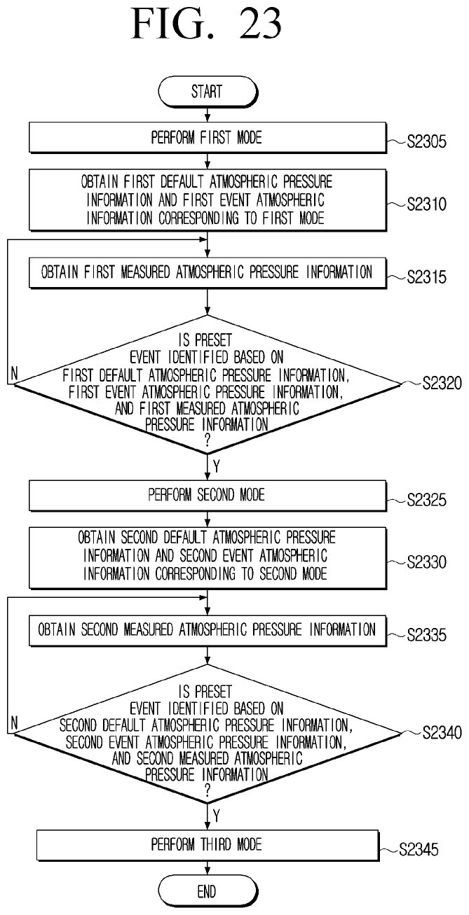

FIG. 23 is a flowchart illustrating an embodiment in which default atmospheric pressure information and event atmospheric pressure information are differently applied according to a performance mode.

Referring to FIG. 23, the electronic mask 100 may perform the first mode in operation S2305.

The electronic mask 100 may obtain the first default atmospheric pressure information and the first event atmospheric pressure information corresponding to the first mode in operation S2310. For example, the first default atmospheric pressure information may be pn1-M1, pn2-M1 of FIG. 24. For example, the first event atmospheric pressure information may be po1-M1, po2-M1 of FIG. 25.

While the electronic mask 100 performs the first mode, the electronic mask 100 may obtain first measured atmospheric pressure information in operation S2315. The electronic mask 100 may determine whether a preset event is identified based on first default atmospheric pressure information, first event atmospheric pressure information, and first measured atmospheric pressure information in operation S2320.

When the preset event is not identified while the first mode is performed in operation S2320-N, the electronic mask 100 may perform operations S2315 to S2320.

When a preset event is identified while the first mode is performed in operation S2320-Y, the electronic mask 100 may perform the second mode in operation S2325.

The electronic mask 100 may obtain the second default atmospheric pressure information corresponding to the second mode and the second event atmospheric pressure information in operation S2330. For example, the second default atmospheric pressure information may be pn1-M2, pn2-M2 of FIG. 24. For example, the second event atmospheric pressure information may be po1-M2, po2-M2 of FIG. 25.

While the electronic mask 100 performs the second mode, the electronic mask 100 may obtain second measured atmospheric pressure information in operation S2335. The electronic mask 100 may determine whether a preset event is identified based on second default atmospheric pressure information, second event atmospheric pressure information, and second measured atmospheric pressure information in operation S2340.

When the preset event is not identified while the second mode is performed in operation S2340-N, the electronic mask 100 may perform operations S2335 to S2340.

When a preset event is identified while the second mode is performed in operation S2340-Y, the electronic mask 100 may perform the third mode in operation S2345.

The electronic mask 100 may obtain the third default atmospheric pressure information corresponding to the third mode and the third event atmospheric pressure information. For example, the third default atmospheric pressure information may be pn1-M3, pn2-M3 of FIG. 24. For example, the third event atmospheric pressure information may be po1-M3, po2-M3 of FIG. 25.

While the electronic mask 100 performs the third mode, the electronic mask 100 may obtain third measured atmospheric pressure information. The electronic mask 100 may determine whether a preset event is identified based on third default atmospheric pressure information, third event atmospheric pressure information, and third measured atmospheric pressure information.

FIG. 24 is a diagram illustrating an embodiment in which default atmospheric pressure information is differently applied according to a performance mode.

The graph 2410 of FIG. 24 illustrates whether default atmospheric pressure information is changed over time. It is assumed that the electronic mask 100 performs the first mode from 0 to t1, performs the second mode from t1 to t2, and performs the third mode from t2.

Default atmospheric pressure information may also be changed when the execution mode is changed from the first mode to the third mode. Since the intensity of the fan motor according to each mode is different, the sensed atmospheric pressure may be different. Even if the user's breathing is constant, an atmospheric pressure sensed may be higher as the fan motor is stronger. Therefore, the electronic mask 100 may change default atmospheric pressure information according to an operating mode.

The first default atmospheric pressure information of the first mode may include default minimum atmospheric pressure (Pn1-M1) and default maximum atmospheric pressure (Pn2-M1).

The second default atmospheric pressure information of the second mode may include default minimum atmospheric pressure (Pn1-M2) and default maximum atmospheric pressure (Pn2-M2).

The third default atmospheric pressure information of the third mode may include default minimum atmospheric pressure (Pn1-M3) and default maximum atmospheric pressure (Pn2-M3).

A solid line 2411 may indicate change in default minimum atmospheric pressure over time.

A solid line 2412 may indicate change in default maximum atmospheric pressure over time.

FIG. 25 is a diagram illustrating an embodiment of differently applying event atmospheric pressure information according to a performance mode.

The graph (2510) of FIG. 25 indicates whether the event atmospheric pressure information is changed over time. It is assumed that the electronic mask 100 performs the first mode from 0 to t1, performs the second mode from t1 to t2, and performs the third mode from t2.

When the execution mode is changed from the first mode to the third mode, the event atmospheric pressure information may also be changed. Since the intensity of the fan motor according to each mode is different, the sensed atmospheric pressure may be different. Even if the breathing is constant, an atmospheric pressure sensed may be higher as the fan motor is stronger. Therefore, the electronic mask 100 may change event atmospheric pressure information according to an operating mode.

The first event atmospheric pressure of the first mode may include inhalation event atmospheric pressure (Po1-M1) and exhalation event atmospheric pressure (Po2-M1).

The second event atmospheric pressure of the second mode may include inhalation event atmospheric pressure (Po1-M2) and exhalation event atmospheric pressure (Po2-M2).

The third event atmospheric pressure of the third mode may include inhalation event atmospheric pressure (Po1-M3) and exhalation event atmospheric pressure (Po2-M3).

A solid line 2511 may indicate change in inhalation event atmospheric pressure over time.

A solid line 2512 may indicate change in exhalation event atmospheric pressure over time.

FIG. 26 is a flowchart illustrating an operation of correcting measurement atmospheric pressure information according to a performance mode.

The operations S2605, S2610, S2615, S2620, S2625, and S2645 of FIG. 26 may correspond to operations S2305, S2310, S2315, S2320, S2325, and S2345 of FIG. 23. Therefore, a duplicate description will be omitted.

After the second mode is performed, the electronic mask 100 may obtain second measured atmospheric pressure information in operation S2630. The electronic mask 100 may obtain second measured atmospheric pressure information corrected by reflecting a correction value (or a correction coefficient) corresponding to the second mode from the second measured atmospheric pressure information in operation S2635.

Assuming that breathing is constant, a sensing atmospheric pressure measured in the second mode may be greater than a sensing atmospheric pressure measured in the first mode. Therefore, the electronic mask 100 may obtain the corrected second measured atmospheric pressure information by subtracting a preset correction value from the sensing atmospheric pressure measured in the second mode.

The electronic mask 100 may determine whether a preset event is identified based on the first default atmospheric pressure information, the first event atmospheric pressure information, and corrected second measured atmospheric pressure in operation S2640.

When a preset event is not identified while operating in the second mode in operation S2640-N, the electronic mask 100 may repeat operations S2630 to S2640.

When a preset event is identified while operating in the second mode in operation S2640-Y, the electronic mask 100 may perform the third mode in operation S2645.

FIG. 27 is a diagram illustrating a preset event (breathing pattern) corresponding to a state or mode.

The table 2710 of FIG. 27 indicates the event type or number of events corresponding to the mode or the state of the electronic mask 100.

When a long exhalation event is identified once for a preset time, the electronic mask 100 may turn on the normal operation mode.

When a long inhalation event is identified once for a preset time, the electronic mask 100 may turn off the normal operation mode.

When a long inhalation event is identified twice for a preset time, the electronic mask 100 may turn off power.

When a short exhalation event is identified once for a preset time, the electronic mask 100 may perform the first mode.

When a short exhalation event is identified twice for a preset time, the electronic mask 100 may perform the second mode.

When a short exhalation event is identified thrice for a preset time, the electronic mask 100 may perform the third mode.

When a short inhalation event is identified thrice during a preset time, the electronic mask 100 may perform a decoding operation. When a short inhalation event is identified thrice during a preset time in a lock state, the electronic mask 100 may change a lock state to an unlock state.

In table 2710 of FIG. 27, it is shown that the first mode, the second mode, and the third mode are determined based on the same event (a short exhalation). According to various embodiments, the electronic mask 100 may determine whether to perform the first mode, the second mode, and the third mode according to the size of the sensing atmospheric pressure.

The table 2720 of FIG. 27 indicates default atmospheric pressure corresponding to each of the first mode, second mode, and third mode. The first default atmospheric pressure corresponding to the first mode may be Pa, the second default atmospheric pressure corresponding to the second mode may be Pb, and the third default atmospheric pressure corresponding to the third mode may be Pc. Here, the second default atmospheric pressure Pb may be greater than the first default atmospheric pressure Pa. The third default atmospheric pressure Pc may be greater than the second default atmospheric pressure Pb.

The default atmospheric pressure may be different from the event atmospheric pressure. The stronger the user breaths exhalation, the electronic mask 100 may perform a mode in which intensity of a fan motor is strong.

In the graph 2510 of FIG. 25, the event atmospheric pressure itself is changed while the mode is performed. In certain embodiments, the table 2720 of FIG. 27 may include information used to immediately determine which mode is to be performed according to the intensity of atmospheric pressure sensed in a specific mode.

For example, in a power saving mode, if sensing atmospheric pressure greater than or equal to the first default atmospheric pressure Pa and less than the second default atmospheric pressure Pb is identified, the electronic mask 100 may perform the first mode.

In addition, in a power saving mode, if sensing atmospheric pressure greater than or equal to the second default atmospheric pressure Pb and less than the third default atmospheric pressure Pc is identified, the electronic mask 100 may perform the second mode.

In addition, in a power saving mode, if sensing atmospheric pressure greater than or equal to the third default atmospheric pressure Pc is identified, the electronic mask 100 may perform the third mode.

According to various embodiments, the event type, number of events, or default pressure (event atmosphere pressure) may be changed according to a user setting.

FIG. 28 is a diagram illustrating a preset event (voice) corresponding to a state or mode.

Table 2810 of FIG. 28 indicates an event voice corresponding to the mode of the electronic mask 100 or the state of the electronic mask 100.

When “normal on” voice is identified during a preset time, the electronic mask 100 may turn on a normal operation mode.

When “normal off” voice is identified during a preset time, the electronic mask 100 may turn off a normal operation mode.

When “power off” voice is identified during a preset time, the electronic mask 100 may turn off power.

When “1 (or mode 1)” voice is identified during a preset time, the electronic mask 100 may perform the first mode.

When “2 (or mode 2)” voice is identified during a preset time, the electronic mask 100 may perform the second mode.

When “3 (or mode 3)” voice is identified during a preset time, the electronic mask 100 may perform the third mode.

When “ABCD” voice is identified during a preset time, the electronic mask 100 may perform a decoding operation. To be specific, in a lock state, when “ABCD” voice is identified, the electronic mask 100 may change a lock state to an unlock state.

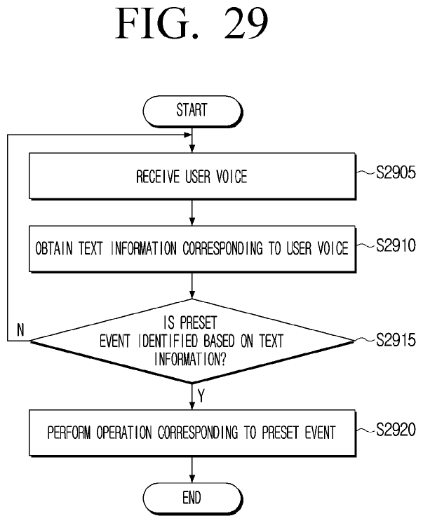

FIG. 29 is a flowchart illustrating an operation of controlling the electronic mask 100 through a user voice.

Referring to FIG. 29, the electronic mask 100 may receive a user voice in

operation S2905. The electronic mask 100 may obtain text information corresponding to the user voice based on a natural language understanding model or a speech recognition model in operation S2910.

The electronic mask 100 may determine whether a preset event is identified based on text information in operation S2915. The preset event may refer to an event in which the preset event voice of FIG. 28 is identified as being included in the user voice.

When a preset event is not identified in operation S2915-N, the electronic mask 100 may repeat operations S2905 to S2915.

When a preset event is identified in operation S2915-Y, the electronic mask 100 may perform an operation corresponding to the terminal device 200 in operation S2920.

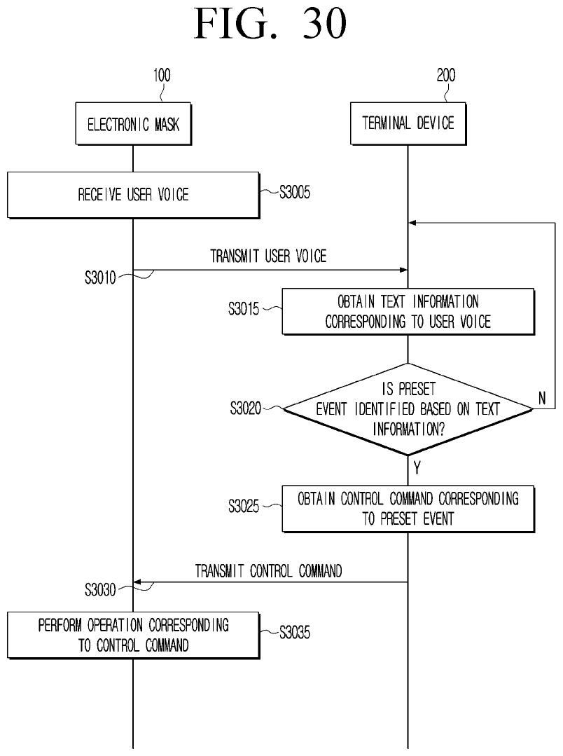

FIG. 30 is a flowchart illustrating an embodiment of analyzing a user voice by using the terminal device 200.

Referring to FIG. 30, the electronic mask 100 may receive a user voice in operation S3005. The electronic mask 100 may transmit a received user voice to the terminal device 200 in operation S3010.

The terminal device 200 may receive a user voice from the electronic mask 100. The terminal device 200 may obtain text information corresponding to a user voice in operation S3015. The terminal device 200 may obtain text information corresponding to a user voice based on a natural language understanding model or a speech recognition model.

The terminal device 200 may determine whether a preset event is identified based on the text information in operation S3020. The preset event may refer to an event in which the preset event voice of FIG. 28 is identified as being included in the user voice.

When a preset event is not identified in operation S3020-N, the terminal device 200 may repeat operations S3015 to S3020.

When a preset event is identified in operation S3020-Y, the terminal device 200 may obtain a control command corresponding to a preset event in operation S3025. The terminal device 200 may transmit a control command to the electronic mask 100 in operation S3030.

The electronic mask 100 may receive a control command from the terminal device 200. The electronic mask 100 may perform an operation corresponding to a control command in operation S3035.

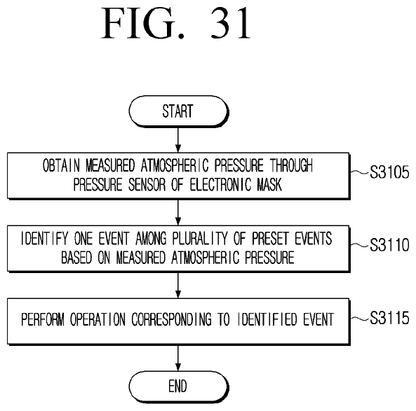

FIG. 31 is a flowchart illustrating a method for controlling the electronic mask 100 according to various embodiments.

Referring to FIG. 31, a method of controlling the electronic mask 100 storing default atmospheric pressure information corresponding to a user and a plurality of preset events corresponding to the default atmospheric pressure information includes obtaining measured atmospheric pressure through a pressure sensor 120 of the electronic mask 100 in operation S3105; identifying one event among the plurality of preset events based on the measured atmospheric pressure in operation S3110; and performing an operation corresponding to the identified event in operation S3115.

The default atmospheric pressure information may include default minimum atmospheric pressure and default maximum atmospheric pressure corresponding to the user, and the identifying the event in operation S3110 may include obtaining first event atmospheric pressure less than the default minimum atmospheric pressure, obtaining second event atmospheric pressure greater than the default maximum atmospheric pressure, and identifying the event by comparing the measured atmospheric pressure with at least one of the first event atmospheric pressure and the second event atmospheric pressure.

The method may further include, based on intensity of the fan module 130 of the electronic mask 100 being changed, changing the default minimum atmospheric pressure, the default maximum atmospheric pressure, or at least one of the first event atmospheric pressure and the second event atmospheric pressure based on the changed intensity of the fan module 130.

The identifying the event in operation S3110 may include, based on the measured atmospheric pressure being less than or equal to the first event atmospheric pressure, identifying an inhalation event among the plurality of preset events.

The identifying the event in operation S3110 may include obtaining a first event time at which the measured atmospheric pressure is sensed to be less than or equal to the first event atmospheric pressure, based on the first event time being less than a threshold time, identifying an inhalation event of a first type, and based on the first event time being greater than or equal to the threshold time, identifying an inhalation event of a second type.

The identifying the event in operation S3110 may include, based on the measured atmospheric pressure being greater than or equal to the second atmospheric pressure, identifying an exhalation event among the plurality of preset events.

The identifying the event in operation S3110 may include obtaining a second event time at which the measured atmospheric pressure is sensed to be greater than or equal to the second event atmospheric pressure, based on the second event time being less than a threshold time, identifying an exhalation event of a first type, and based on the second event time being greater than or equal to the threshold time, identifying an exhalation event of a second type.

The identifying the event in operation S3110 may include identifying number of events that the event is identified during a preset time, and identifying the event based on the number of events.

The default atmospheric pressure information may include a default breathing time, and the identifying the event in operation S3110 may include, based on an inhalation event being identified, obtaining a breathing time of the user based on a time interval corresponding to an extreme minimum point, based on an exhalation event being identified, obtaining a breathing time of the user based on a time interval corresponding to an extreme maximum point, and identifying the event based on comparing the default breathing time and the breathing time.

The method may further include receiving a user input to change setting information of the electronic mask 100 from the terminal device 200 and changing setting information of the electronic mask 100 based on the received user input.

The control method of the electronic mask 100 like FIG. 31 may be executed by the electronic mask 100 having a configuration of FIG. 2 or FIG. 3, or may be executed by the electronic mask 100 having other configurations.

The methods according to various embodiments may be implemented as a format of software or application installable to a related art electronic apparatus or an electronic mask.

The methods according to various embodiments may be implemented by software upgrade of a related art electronic apparatus or an electronic mask, or hardware upgrade only.

Also, various embodiments of the disclosure described above may be performed through an embedded server provided in an electronic apparatus or an electronic mask, or through an external server of at least one of an electronic apparatus and an electronic mask and a display device.

Meanwhile, various embodiments of the disclosure may be implemented in software, including instructions stored on machine-readable storage media readable by a machine (e.g., a computer). An apparatus may call instructions from the storage medium, and execute the called instruction, including an image processing apparatus (for example, image processing apparatus A) according to the disclosed embodiments. When the instructions are executed by a processor, the processor may perform a function corresponding to the instructions directly or using other components under the control of the processor. The instructions may include a code generated by a compiler or a code executable by an interpreter. A machine-readable storage medium may be provided in the form of a non-transitory storage medium. Herein, the “non-transitory” storage medium may not include a signal but is tangible, and does not distinguish the case in which a data is semi-permanently stored in a storage medium from the case in which a data is temporarily stored in a storage medium.

According to an embodiment, the method according to the above-described embodiments may be included in a computer program product. The computer program product may be traded as a product between a seller and a consumer. The computer program product may be distributed online in the form of machine-readable storage media (e.g., compact disc read only memory (CD-ROM)) or through an application store (e.g., Play Store™) or distributed online directly. In the case of online distribution, at least a portion of the computer program product may be at least temporarily stored or temporarily generated in a server of the manufacturer, a server of the application store, or a machine-readable storage medium such as memory of a relay server.

According to various embodiments, the respective elements (e.g., module or program) of the elements mentioned above may include a single entity or a plurality of entities. According to the embodiments, at least one element or operation from among the corresponding elements mentioned above may be omitted, or at least one other element or operation may be added. Alternatively or additionally, a plurality of components (e.g., module or program) may be combined to form a single entity. In this case, the integrated entity may perform functions of at least one function of an element of each of the plurality of elements in the same manner as or in a similar manner to that performed by the corresponding element from among the plurality of elements before integration. The module, a program module, or operations executed by other elements according to variety of embodiments may be executed consecutively, in parallel, repeatedly, or heuristically, or at least some operations may be executed according to a different order, may be omitted, or the other operation may be added thereto.

While various embodiments have been illustrated and described with reference to various embodiments, the disclosure is not limited to specific embodiments or the drawings, and it will be understood by those of ordinary skill in the art that various changes in form and details may be made therein without departing from the spirit and scope of the disclosure, including the appended claims and their equivalents.

Although the present disclosure has been described with various embodiments, various changes and modifications may be suggested to one skilled in the art. It is intended that the present disclosure encompass such changes and modifications as fall within the scope of the appended claims.

What is claimed is:

1. An electronic mask comprising:
a memory configured to store default atmospheric pressure information corresponding to a user and a plurality of preset events corresponding to the default atmospheric pressure information;
a pressure sensor configured to measure atmospheric pressure inside the electronic mask;
a fan module configured to introduce outside air to inside of the electronic mask or discharge inside air to outside of the electronic mask; and
at least one processor configured to:
obtain the measured atmospheric pressure through the pressure sensor,
identify an event among the plurality of preset events based on the measured atmospheric pressure, and
control the fan module to perform an operation corresponding to the identified event.

2. The electronic mask of claim 1, wherein:
the default atmospheric pressure information comprises a default minimum atmospheric pressure and a default maximum atmospheric pressure corresponding to the user, and
the at least one processor is configured to:
obtain a first event atmospheric pressure less than the default minimum atmospheric pressure,
obtain a second event atmospheric pressure greater than the default maximum atmospheric pressure, and
identify the event by comparing the measured atmospheric pressure with at least one of the first event atmospheric pressure and the second event atmospheric pressure.

3. The electronic mask of claim 2, wherein the at least one processor is configured to:
in response to an intensity of the fan module changing, change the default minimum atmospheric pressure, the default maximum atmospheric pressure, or at least one of the first event atmospheric pressure and the second event atmospheric pressure based on the changed intensity of the fan module.

4. The electronic mask of claim 2, wherein the at least one processor is configured to, based on the measured atmospheric pressure being less than or equal to the first event atmospheric pressure, identify an inhalation event among the plurality of preset events.

5. The electronic mask of claim 4, wherein the at least one processor is configured to:
obtain a first event time at which the measured atmospheric pressure is less than or equal to the first event atmospheric pressure,
based on the first event time being less than a threshold time, identify the inhalation event as a first type, and
based on the first event time being greater than or equal to the threshold time, identify the inhalation event as a second type.

6. The electronic mask of claim 2, wherein the at least one processor is configured to, based on the measured atmospheric pressure being greater than or equal to the second atmospheric pressure, identify an exhalation event among the plurality of preset events.

7. The electronic mask of claim 6, wherein the at least one processor is configured to:
obtain a second event time at which the measured atmospheric pressure is greater than or equal to the second event atmospheric pressure,
based on the second event time being less than a threshold time, identify the exhalation event as a first type, and
based on the second event time being greater than or equal to the threshold time, identify the exhalation event as a second type.

8. The electronic mask of claim 1, wherein the at least one processor is configured to:
identify a number of events based on an amount the event is identified during a preset time, and
identify the event based on the number of events.

9. The electronic mask of claim 1, wherein:
the default atmospheric pressure information comprises a default breathing time, and
the at least one processor is configured to:
based on an inhalation event being identified, obtain a breathing time of the user based on a first time interval corresponding to an extreme minimum point,
based on an exhalation event being identified, obtain the breathing time of the user based on a second time interval corresponding to an extreme maximum point, and
identify the event based on comparing the default breathing time and the breathing time.

10. The electronic mask of claim 1, comprising:
a communication interface,
wherein the at least one processor is configured to:
receive a user input to change setting information of the electronic mask from a terminal device through the communication interface, and
change setting information of the electronic mask based on the received user input.

11. A method of controlling an electronic mask storing default atmospheric pressure information corresponding to a user and a plurality of preset events corresponding to the default atmospheric pressure information, the method comprising:
obtaining a measured atmospheric pressure through a pressure sensor of the electronic mask;
identifying an event among the plurality of preset events based on the measured atmospheric pressure; and
performing an operation corresponding to the identified event.

12. The method of claim 11, wherein:
the default atmospheric pressure information comprises a default minimum atmospheric pressure and a default maximum atmospheric pressure corresponding to the user,
wherein identifying the event comprises:
obtaining a first event atmospheric pressure less than the default minimum atmospheric pressure,
obtaining a second event atmospheric pressure greater than the default maximum atmospheric pressure, and
identifying the event by comparing the measured atmospheric pressure with at least one of the first event atmospheric pressure and the second event atmospheric pressure.

13. The method of claim 12, further comprising:
in response to an intensity of a fan module changing, changing the default minimum atmospheric pressure, the default maximum atmospheric pressure, or at least one of the first event atmospheric pressure and the second event atmospheric pressure based on the changed intensity of the fan module.

14. The method of claim 12, wherein the identifying the event comprises, based on the measured atmospheric pressure being less than or equal to the first event atmospheric pressure, identifying an inhalation event among the plurality of preset events.

15. The method of claim 14, wherein the identifying the event comprises:
obtaining a first event time at which the measured atmospheric pressure is less than or equal to the first event atmospheric pressure,
based on the first event time being less than a threshold time, identifying the inhalation event as a first type, and
based on the first event time being greater than or equal to the threshold time, identifying the inhalation event as a second type.

16. The method of claim 12, further comprising:
based on the measured atmospheric pressure being greater than or equal to the second atmospheric pressure, identifying an exhalation event among the plurality of preset events.

17. The method of claim 16, further comprising:
obtaining a second event time at which the measured atmospheric pressure is greater than or equal to the second event atmospheric pressure,
based on the second event time being less than a threshold time, identifying the exhalation event as a first type, and
based on the second event time being greater than or equal to the threshold time, identifying the exhalation event as a second type.

18. The method of claim 11, further comprising:
identifying a number of events based on an amount the event is identified during a preset time, and
identifying the event based on the number of events.

19. The method of claim 11, wherein:
the default atmospheric pressure information comprises a default breathing time, and
the method further comprises:
based on an inhalation event being identified, obtaining a breathing time of the user based on a first time interval corresponding to an extreme minimum point,
based on an exhalation event being identified, obtaining the breathing time of the user based on a second time interval corresponding to an extreme maximum point, and
identifying the event based on comparing the default breathing time and the breathing time.

20. The method of claim 11, further comprising:
receiving a user input to change setting information of the electronic mask from a terminal device through a communication interface of the electronic mask, and
changing setting information of the electronic mask based on the received user input.
